SEQUENCE LISTING (9 sequences)

SEQ ID NO: 1 - DNA, 3410 bp, Corynebacterium glutamicum
  CDS (1001)..(2407): lpdA gene
acggtgatcgctttggcacttgcctgtgcactgaacaccatcgaactgcccatcggcattcgggtgattttccagccggcagaagaagtcatgactggtggcgcaacggacgtcattgcccacggtggccttgatggtgtggatgcgatttacgccatccacgttgaacccaaattgaaggtcggtcgcgtcggtgtacgcgctggcgcgattacttctgcctcagatgtgatcgaaatcagagtcaagggtgaaggaggacatagcgcacgtccacacctctccgctgatgttgtttacgccttgagcaaattggtcgttgatcttcccggtttgctgtccaggcgcgtcgatccacgcaccggcaccgtgcttgttttcggcaccatcaacgccggctatgcgcccaacgcgatcccagattccggcatcgtgtcaggcaccttgcgtacagccgacatctctacctggcgtgacatgcgtccgcttatctctgagctggtggaacaggtgctcgcacccaccggagtcacccatgaactgatctacaatccgggtgttccaccagtgcttaacgacgatgtcgccaccgctttgttggcaagcgcagcacgcgacatggacacacaatctgttgtccaagcgccgcagtcatccggtggagaagacttctcgtggtaccttgaacacgtcccaggatcaatggcccggttgggttgctggccggggcacggacccaagcaagacctccatcaaagtgacctggttgtggatgagcgagccatcggagttggcgtcaggctctttggctcccttgtgcagcagtacagtagccgatctgaagctttcttaaattcctaatgggggtagtgtgtagggctggctctaaattgctgacacgtcacccaggctaattcagcagtaatcatttagacttggaaccgcttaccagtggtttcaacaatgcattcacccagctcacacgtgtggaggtgccttaatggcaaagaggatcgtaattatcggcggtggacctgcaggctatgaagccgcactcgcaggcgctaaatacggtgcagaagttaccgttattgaagatgtcggagttggcggatccgcagtcaccatggactgtgtaccttcaaagtccttcatcgctggtaccggtatcaaaaccgacctccgacgtgctgatgacatgggacttaaccgtgggcttggaaaagcacacctagaaatcgatgcactgaacatccgtgtgaaggaccttgcgaaagcacagtccgaagatatcttgggccagctgcagcgctcagatgtccgcatgattaacggtgtgggccgctttgatgattacaacaccaagcaaaccacccactacattaaagtcacccacagcgatggctccgaagaaaccgttgagtgcgatctggtgctggttgcaactggtgcaaccccccgcattcttaaaggtgcagagccagacggcgagcgcatcttgacctggcgtcaggtctacgacattgaagaactccccacccaccttatcgtggttggttccggtgtgaccggtgcggaatttgtctctgcgtttgctgaactcggcgtcaaagtcaccatggtggcatcccgtgaccgcattttgcctcacgatgacgcagatgccgcagacgtgctggaaaccgttctggctgagcgcggagtatccctggaaaagcatgcccgcgtggagtctgtcacccgcaccgaagacggtggcgtgtgtgttcgcactgctgacggacgagaaatctacggttctcacgcgttgatgactgttggttccattccaaacacggcagatcttggcctggagaacatcggtgttgagctggcaccatccggccatatcaaggttgaccgcgtctcccgcaccaacatccccggtgtgtacgcagcaggtgactgtactgacctattcccactggcgtccgttgcagcgatgcagggccgtatcgccatgtatcacgcactcggtgaaggcgtgagccccatccgtttgaagactgttgccaccgcagtgtttacccgcccagagatcgcagcagtaggtatcacccatgcacaagttgattccggcgaagtgtctgctcgcgtgattgtgcttcctttggctactaacccacgcgccaagatgcgttccctgcgccacggttttgtgaagctgttctgccgccgtaactctggcctgatcatcggtggtgtcgtggtggcaccgaccgcgtctgagctgatcctaccgatcgctgtggcagtgaccaaccgtctgacagttgctgatctggctgataccttcgcggtgtacccatcattgtcaggttcgattactgaagcagcacgtcagctggttcaacatgatgatctaggctaatttttctgagtcttagattttgagaaaacccaggattgctttgtgcactcctgggttttcactttgttaagcagttttggggaaaagtgcaaagtttgcaaagtttagaaatattttaagaggtaagatgtctgcaggtggaagcgtttaaatgcgttaaacttggccaaatgtggcaacctttgcaaggtgaaaaactggggcggggttagatcctggggggtttatttcattcactttggcttgaagtcgtgcaggtcaggggagtgttgcccgaaaacattgagaggaaaacaaaaaccgatgtttgattgggggaatcgggggttacgatactaggacgcagtgactgctatcacccttggcggtctcttgttgaaaggaataattactctagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctt

SEQ ID NO: 2 - protein, 469 aa, Corynebacterium glutamicum
MAKRIVIIGGGPAGYEAALAGAKYGAEVTVIEDVGVGGSAVTMDCVPSKSFIAGTGIKTDLRRADDMGLNRGLGKAHLEIDALNIRVKDLAKAQSEDILGQLQRSDVRMINGVGRFDDYNTKQTTHYIKVTHSDGSEETVECDLVLVATGATPRILKGAEPDGERILTWRQVYDIEELPTHLIVVGSGVTGAEFVSAFAELGVKVTMVASRDRILPHDDADAADVLETVLAERGVSLEKHARVESVTRTEDGGVCVRTADGREIYGSHALMTVGSIPNTADLGLENIGVELAPSGHIKVDRVSRTNIPGVYAAGDCTDLFPLASVAAMQGRIAMYHALGEGVSPIRLKTVATAVFTRPEIAAVGITHAQVDSGEVSARVIVLPLATNPRAKMRSLRHGFVKLFCRRNSGLIIGGVVVAPTASELILPIAVAVTNRLTVADLADTFAVYPSLSGSITEAARQLVQHDDLG

SEQ ID NO: 3 - DNA, 1706 bp, Corynebacterium glutamicum
  promoter (612)..(1100): Promoter Pg3 with RBS
cccggggatccatgcgcccaacgcgatcccagattccggcatcgtgtcaggcaccttgcgtacagccgacatctctacctggcgtgacatgcgtccgcttatctctgagctggtggaacaggtgctcgcacccaccggagtcacccatgaactgatctacaatccgggtgttccaccagtgcttaacgacgatgtcgccaccgctttgttggcaagcgcagcacgcgacatggacacacaatctgttgtccaagcgccgcagtcatccggtggagaagacttctcgtggtaccttgaacacgtcccaggatcaatggcccggttgggttgctggccggggcacggacccaagcaagacctccatcaaagtgacctggttgtggatgagcgagccatcggagttggcgtcaggctctttggctcccttgtgcagcagtacagtagccgatctgaagctttcttaaattcctaatgggggtagtgtgtagggctggctctaaattgctgacacgtcacccaggctaattcagcagtaatcatttagacttggaaccgcttaccagtggtttcaacaatgcattcacccagctcacacgtgtggaggtgccttaagtacttgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgctgcgaaatctttgtttccccgctaaagttgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatggcaaagaggatcgtaattatcggcggtggacctgcaggctatgaagccgcactcgcaggcgctaaatacggtgcagaagttaccgttattgaagatgtcggagttggcggatccgcagtcaccatggactgtgtaccttcaaagtccttcatcgctggtaccggtatcaaaaccgacctccgacgtgctgatgacatgggacttaaccgtgggcttggaaaagcacacctagaaatcgatgcactgaacatccgtgtgaaggaccttgcgaaagcacagtccgaagatatcttgggccagctgcagcgctcagatgtccgcatgattaacggtgtgggccgctttgatgattacaacaccaagcaaaccacccactacattaaagtcacccacagcgatggctccgaagaaaccgttgagtgcgatctggtgctggttgcaactggtgcaaccccccgcattcttaaaggtgcagagccagacggcgagcgcatcttgacctggcgtcaggtctacgacattgaagaactccccacccaccttatcgtggttggttccggtgtgaccggtgcggaatttgtctctgcgtttgctgaatctaga

SEQ ID NO: 4 - DNA, 46 bp, Corynebacterium glutamicum
  misc_feature (1)..(46): Primer lpdA_pZ8-1-1
ggtggtgaattcaaaggaggacaaccatggcaaagaggatcgtaat

SEQ ID NO: 5 - DNA, 32 bp, Corynebacterium glutamicum
  misc_feature (1)..(32): Primer lpdA_pZ8-1-2
ggtggtgtcgacttagcctagatcatcatgtt

SEQ ID NO: 6 - DNA, 1448 bp, Corynebacterium glutamicum
  CDS (27)..(1433): lpdA gene
ggtggtgaattcaaaggaggacaaccatggcaaagaggatcgtaattatcggcggtggacctgcaggctatgaagccgcactcgcaggcgctaaatacggtgcagaagttaccgttattgaagatgtcggagttggcggatccgcagtcaccatggactgtgtaccttcaaagtccttcatcgctggtaccggtatcaaaaccgacctccgacgtgctgatgacatgggacttaaccgtgggcttggaaaagcacacctagaaatcgatgcactgaacatccgtgtgaaggaccttgcgaaagcacagtccgaagatatcttgggccagctgcagcgctcagatgtccgcatgattaacggtgtgggccgctttgatgattacaacaccaagcaaaccacccactacattaaagtcacccacagcgatggctccgaagaaaccgttgagtgcgatctggtgctggttgcaactggtgcaaccccccgcattcttaaaggtgcagagccagacggcgagcgcatcttgacctggcgtcaggtctacgacattgaagaactccccacccaccttatcgtggttggttccggtgtgaccggtgcggaatttgtctctgcgtttgctgaactcggcgtcaaagtcaccatggtggcatcccgtgaccgcattttgcctcacgatgacgcagatgccgcagacgtgctggaaaccgttctggctgagcgcggagtatccctggaaaagcatgcccgcgtggagtctgtcacccgcaccgaagacggtggcgtgtgtgttcgcactgctgacggacgagaaatctacggttctcacgcgttgatgactgttggttccattccaaacacggcagatcttggcctggagaacatcggtgttgagctggcaccatccggccatatcaaggttgaccgcgtctcccgcaccaacatccccggtgtgtacgcagcaggtgactgtactgacctattcccactggcgtccgttgcagcgatgcagggccgtatcgccatgtatcacgcactcggtgaaggcgtgagccccatccgtttgaagactgttgccaccgcagtgtttacccgcccagagatcgcagcagtaggtatcacccatgcacaagttgattccggcgaagtgtctgctcgcgtgattgtgcttcctttggctactaacccacgcgccaagatgcgttccctgcgccacggttttgtgaagctgttctgccgccgtaactctggcctgatcatcggtggtgtcgtggtggcaccgaccgcgtctgagctgatcctaccgatcgctgtggcagtgaccaaccgtctgacagttgctgatctggctgataccttcgcggtgtacccatcattgtcaggttcgattactgaagcagcacgtcagctggttcaacatgatgatctaggctaagtcgacaccacc

SEQ ID NO: 7 - protein, 469 aa, Corynebacterium glutamicum; identical to SEQ ID NO: 2

SEQ ID NO: 8 - DNA, 20 bp, Corynebacterium glutamicum
aacacgtcccaggatcaatg

SEQ ID NO: 9 - DNA, 20 bp, Corynebacterium glutamicum
tttcgcaaggtccttcacac

(19) United States
(12) Patent Application Publication
(10) Pub. No.: US 2016/0265072 A2
(43) Pub. Date: Sep. 15, 2016

(54) MICROORGANISM AND METHOD FOR PRODUCTION OF AMINO ACIDS BY FERMENTATION

(71) Applicant: EVONIK DEGUSSA GMBH, Essen (DE)

(72) Inventors: Frederik Walter, Bielefeld (DE); Marcus Persicke, Bielefeld (DE); Jorn Kalinowski, Bielefeld (DE); Andrea Huser, Bielefeld (DE); Wilfried Claes, Bielefeld (DE); Alexander Reth, Bielefeld (DE)

(21) Appl. No.: 14/762,840

(22) PCT Filed: Jan. 13, 2014
(86) PCT No.: PCT/EP2014/050484

§ 371 Date: Jul. 23, 2015

(51) Int. Cl. C12R 1/15; C12P 13/08; C12P 13/12

(52) U.S. Cl. CPC C12R 1/15; C12P 13/12; C12P 13/08

(57) ABSTRACT

The invention relates to a microorganism that produces and/or secretes an organic chemical compound, wherein the microorganism has an increased expression, compared to the respective starting strain, of a polypeptide LpdA with the activity of a transhydrogenase; and a process for producing an organic chemical compound using the microorganism according to the invention.

The invention relates to a microorganism that produces and/or secretes an organic chemical compound, wherein the microorganism has an increased activity of a transhydrogenase (LpdA protein, gene product of the lpdA gene); and a process for producing an organic chemical compound using the microorganism according to the invention.

The present invention came about within the scope of a project promoted by the Federal Ministry for Education and Research (BMBF) (Grant number 0313917A).

### PRIOR ART

Organic chemical compounds, including in particular amino acids, organic acids, vitamins, nucleosides and nucleotides, find application in human medicine, in the pharmaceutical industry, in the food industry and quite especially in animal nutrition.

A great many of these compounds, for example L-lysine, are produced by fermentation by strains of coryneform bacteria, especially Corynebacterium glutamicum, or by strains of the Enterobacteriaceae family, especially Escherichia coli. Owing to the considerable economic importance, there are constant efforts to improve the production processes. Process improvements can relate to technical measures in fermentation, for example stirring and supplying oxygen, or to the composition of the nutrient media, for example the sugar concentration during fermentation, or to work-up to the product form for example by ion exchange chromatography or to the intrinsic performance properties of the microorganism itself.

In wild-type strains, biosynthesis pathways of amino acids are subject to strict metabolic control, which ensures that the amino acids are only produced at the level required for the cell's own needs. An important requirement for efficient production processes is therefore for suitable microorganisms to be available, which in contrast to wild-type organisms have capacity for producing the desired amino acid that is dramatically increased beyond their own requirements (overproduction).

Improvement of the performance properties of these microorganisms employs methods of mutagenesis, selection and picking mutants. In this way strains are obtained that are resistant to antimetabolites or are auxotrophic for metabolites of regulatory importance and that produce L-amino acids. A known antimetabolite is the lysine analogue S-(2-aminoethyl)-L-cysteine (AEC).

For some years, methods of recombinant DNA technology have also been used for improving L-amino acid-producing strains of the genus Corynebacterium, especially Corynebacterium glutamicum, or of the genus Escherichia, especially Escherichia coli, by modifying or strengthening or weakening individual amino acid biosynthesis genes and investigating the effect on amino acid production.

The nucleotide sequences of the chromosomes of numerous bacteria are known.

The nucleotide sequence of the genome of Corynebacterium glutamicum ATCC13032 is described in Ikeda and Nakagawa (Applied Microbiology and Biotechnology 62, 99-109 (2003)), in EP 1 108 790 and in Kalinowski et al. (Journal of Biotechnology 104(1-3), (2003)).

The nucleotide sequence of the genome of Corynebacterium glutamicum R is described in Yukawa et al. (Microbiology 153(4): 1042-1058 (2007)).

The nucleotide sequence of the genome of Corynebacterium efficiens is described in Nishio et al. (Genome Research. 13 (7), 1572-1579 (2003)).

The nucleotide sequence of the genome of Corynebacterium diphtheriae NCTC 13129 was described by Cerdeno-Tarraga et al. (Nucleic Acids Research 31 (22), 6516-6523 (2003)).

The nucleotide sequence of the genome of Corynebacterium jeikeium was described by Tauch et al. (Journal of Bacteriology 187 (13), 4671-4682 (2005)).

The nucleotide sequences of the genome of Corynebacterium glutamicum are also available in the data bank of the National Center for Biotechnology Information (NCBI) of the National Library of Medicine (Bethesda, Md., USA), in the DNA Data Bank of Japan (DDBJ, Mishima, Japan) or in the nucleotide sequence database of the European Molecular Biology Laboratories (EMBL, Heidelberg, Germany or Cambridge, UK).

Various aspects of the production of L-amino acids by fermentation are summarized in R. Faurie and J. Thommel, Advances in Biochemical Engineering Biotechnology, Vol. 79 (Springer-Verlag, Berlin, Heidelberg (Germany) 2003).

In the production of the aforementioned organic chemical compounds using microorganisms, each of these compounds is produced in a biosynthesis pathway in the cell of a microorganism. One of the important coenzymes that are essential for the function of important enzymes in the biosynthesis system is reduced nicotinamide-adenine dinucleotide phosphate (called NADPH hereinafter).

The relationship between NADPH and the production of organic chemical compounds using microorganisms is explained for example in EP0733712B.

Nicotinamide dinucleotide transhydrogenase (called simply “transhydrogenase” hereinafter) is known to be one of the enzymes responsible for the production of NADPH. It is known that this enzyme is present in various organisms, including in microorganisms of the genus Escherichia. In Escherichia coli, a typical microorganism of the genus Escherichia, purification of transhydrogenase (David M. Clarke and Philip D. Bragg, Eur. J. Biochem., 149, 517-523 (1985)), cloning of a gene encoding it (David M. Clarke and Philip D. Bragg, J. Bacteriology., 162, 367-373 (1985)) and determination of the nucleotide sequence of the gene (David M. Clarke, Tip W. Loo, Shirley Gillam, and Philip D. Bragg, Eur. J. Biochem. 158, 647-653 (1986)) were carried out, and the presence of the enzyme was demonstrated. However, the physiological function of the enzyme is still almost unknown. This is typically demonstrated by the fact that variants lacking the enzyme do not display any phenotypical expression.

Makoto Ishimoto “Metabolic Maps”, Jul. 25, 1971 (Kyoritsu Suppan Co., Ltd.), pages 30-32, discloses that the reducing activity of NADH or NADPH is necessary for the biosynthesis of several amino acids. This document does not, however, disclose the conversion of NADH to NADPH for the biosynthesis of a target substance.

Bunji Maruo, Nobuo Tamiya (authors) “Enzyme Handbook”, Mar. 1, 1983 (01.03.83), Asakura Shoten, p. 132-133 and Arch. Biochem. Biophys. Vol. 176, No. 1, 1976, Wermuth B et al. “Pyridine nucleotide transhydrogenase from Pseudomonas aeruginosa purification by affinity chromatography and physiochemical properties”, p. 136-143, in each case disclose an enzyme that is able to convert NADH to NADPH and vice versa. None of these documents relates to the production of a target substance or an increased productivity for NADPH, that could be used for producing a target substance.

E. coli contains both a soluble and a membrane-bound pyridine-nucleotide transhydrogenase. The soluble pyridine-nucleotide transhydrogenase is the gene product SthA (Sth, UdhA); its primary physiological role appears to be reoxidation of NADPH. The membrane-bound proton-transferring pyridine-nucleotide transhydrogenase is the PntAB gene product; PntAB is an important source of NADPH (Sauer et al., The Journal of Biological Chemistry 279(8) (2004)).

Anderlund et al. (Applied and Environmental Microbiology 56(6) (1999)) investigated the physiological effect of the expression of membrane-bound transhydrogenase (pntA and pntB genes) from Escherichia coli in recombinant Saccharomyces cerevisiae.

Against this background, the problem to be solved by the present invention is to provide a microorganism that is improved relative to the microorganisms described in the prior art, methods of producing said microorganism and methods using said microorganism for the overproduction of organic chemical compounds, wherein the improvement relates to factors such as the concentration of the overproduced compound attained intracellularly, the yield of the overproduced compound after processing the culture, the growth properties of the microorganism as well as the time and number of process steps required for overproduction and processing of the compound and the resource requirement of the process, for example with respect to time, energy and amount of strains and educts used.

These and other problems are solved by the subject matter of the present application and especially by the subject matter of the independent claims, wherein embodiments arise from the subclaims.

In a first aspect, the problem to be solved by the application is solved by a process for producing an organic chemical compound by fermentation using a microorganism containing the steps:

a. fermentation of a microorganism producing an organic chemical compound,
wherein, in the microorganism, a polynucleotide is overexpressed that codes for a polypeptide whose amino acid sequence is at least 80%, at least 85%, at least 90%, at least 95%, at least 98% or at least 99% identical to the amino acid sequence of SEQ ID NO:2,
and wherein the fermentation takes place in a suitable fermentation medium with formation of a fermentation broth and
b. enrichment (accumulation) of the organic chemical compound in the fermentation broth from a).

In the context of the present invention, a microorganism producing an organic chemical compound means a microorganism that produces the organic chemical compound beyond what is necessary for maintaining the viability of the microorganism.

In the context of the present invention, enrichment, or accumulation, of the organic chemical compound in the fermentation broth means both the enrichment/accumulation of the organic chemical compounds in the cells of the microorganism and the secretion of the organic chemical compound into the nutrient medium surrounding the microorganism, i.e. into the fermentation broth.

In another aspect, the problem to be solved by the application is solved with a microorganism that produces an organic chemical compound, wherein a polynucleotide that codes for a polypeptide whose amino acid sequence is at least 80%, at least 85%, at least 90%, at least 95%, at least 98% or at least 99% identical to the amino acid sequence of SEQ ID NO:2, is overexpressed in the microorganism.

In one embodiment of the process according to the invention, for producing an organic chemical compound, or the microorganism according to the invention, the encoded polypeptide, whose amino acid sequence is at least 80%, at least 85%, at least 90%, at least 95%, at least 98% or at least 99% identical to the amino acid sequence of SEQ ID NO:2, has the activity of a transhydrogenase.

The activity of a transhydrogenase is detected by the enzyme test according to Argyrou et al. (2004). In this test, purified transhydrogenase is investigated with respect to the NAD(P)H-dependent reduction of 5-HNQ, an artificial electron acceptor.

The microorganisms or strains (starting strains) used for the measures for overexpression of the polypeptide disclosed in the claims preferably already possess the capacity for enriching the desired organic chemical compound in the cell or for secreting it in the surrounding nutrient medium and accumulating it there.

The advantage of the present invention is that the yield of product in biotechnological production processes, e.g. amino acid production, is further increased. With the process according to the invention, or by using the microorganisms according to the invention, the microbial production of organic chemical compounds can be further increased.

Therefore the process according to the invention for producing an organic chemical compound is also characterized in that the production of the organic chemical compound is increased by at least 0.5%, at least 1%, at least 1.5% or at least 2% relative to the fermentation of a microorganism in which the polynucleotide that codes for a polypeptide whose amino acid sequence is at least 80% identical to the amino acid sequence of SEQ ID NO:2, is not overexpressed. Surprisingly, a transhydrogenase in C. glutamicum was identified from a high sequence identity to a protein with the activity of a transhydrogenase (LpdA protein) in Mycobacterium tuberculosis. The increase in expression of the transhydrogenase led to increased production of an organic chemical compound when using a corresponding production strain.

In a preferred embodiment of the process according to the invention/of the microorganism according to the invention, the polynucleotide that codes for a polypeptide whose amino acid sequence is at least 80%, at least 85%, at least 90%, at least 95%, at least 98% or at least 99%, or 100% identical to the amino acid sequence of SEQ ID NO:2 is derived from Corynebacterium glutamicum. Especially preferably this encoded polypeptide has the activity of a transhydrogenase.

The microorganism according to the invention has, compared to the respective starting strain, increased expression of a polynucleotide coding for a polypeptide with an amino acid sequence displaying identity of 80% or more to the amino acid sequence shown in SEQ ID NO:2.

Preferred embodiments include variants that are at least 80%, at least 85%, at least 90%, at least 95%, at least 98% or at least 99% identical to the amino acid sequence shown in SEQ ID NO:2, i.e. wherein at least 80%, at least 85%, at least 90%, at least 95%, at least 98% or at least 99% of the amino acid positions are identical to the amino acid sequence shown in SEQ ID NO:2. The percentage identity is preferably calculated over the whole length of the amino acid or nucleic acid region.

In a preferred embodiment the microorganism has, compared to the respective starting strain, increased expression of a polynucleotide coding for a polypeptide with an amino acid sequence according to SEQ ID NO:2.

As reduction equivalents in the form of NADPH are required for the biosynthesis of organic chemical compounds, the improved provision with reduction equivalents is advantageous.

An “organic chemical compound” means, for the measures of the invention, a vitamin, for example thiamine (vitamin B1), riboflavin (vitamin B2), cyanocobalamin (vitamin B12), folic acid (vitamin M), tocopherol (vitamin E) or nicotinic acid/nicotinamide, a nucleoside or nucleotide, for example S-adenosylmethionine, inosine 5′-monophosphoric acid and guanosine 5′-monophosphoric acid, L-amino acids, organic acids or also an amine, for example cadaverine. L-amino acids and products containing them are preferably produced.

The organic chemical compound that is produced and/or secreted by the microorganism according to the invention is preferably selected from the group comprising vitamin, nucleoside or nucleotide, L-amino acids, organic acids and amine.

The term L-amino acids comprises the proteinogenic amino acids, plus L-ornithine and L-homoserine. Proteinogenic L-amino acids are to be understood as the L-amino acids that occur in natural proteins, i.e. in proteins of microorganisms, plants, animals and humans. The proteinogenic amino acids include L-aspartic acid, L-asparagine, L-threonine, L-serine, L-glutamic acid, L-glutamine, L-glycine, L-alanine, L-cysteine, L-valine, L-methionine, L-isoleucine, L-leucine, L-tyrosine, L-phenylalanine, L-histidine, L-lysine, L-tryptophan, L-arginine, L-proline and optionally L-selenocysteine and L-pyrrolysine.

The term organic acid comprises α-keto acids, especially an α-keto acid selected from the group α-ketoisocaproic acid, α-ketovaleric acid and α-keto-β-methylvaleric acid.

Especially preferably, the organic chemical compound is selected from the group proteinogenic L-amino acid, L-ornithine and L-homoserine as well as α-ketoisocaproic acid, α-ketovaleric acid and α-keto-β-methylvaleric acid. Especially preferably the proteinogenic L-amino acid is selected from the group L-threonine, L-lysine, L-methionine, L-valine, L-proline, L-glutamate, L-leucine and L-isoleucine, especially L-lysine, L-methionine and L-valine, quite especially L-lysine and L-methionine.

If amino acids or L-amino acids are mentioned in the following, the term also comprises the salts thereof, for example lysine monohydrochloride or lysine sulphate in the case of the amino acid L-lysine. Similarly, the term α-keto acid also comprises its salts, such as in the case of α-ketoisocaproic acid (KIC): calcium KIC, potassium KIC or sodium KIC.

The microorganism is preferably selected from the group bacteria, yeast and fungi, among the bacteria especially preferably from the family Corynebacteriaceae or the family Enterobacteriaceae, wherein the genus Corynebacterium or the genus Escherichia is quite especially preferred and from the stated genera, the species Corynebacterium glutamicum or the species Escherichia coli is especially preferred.

In another preferred embodiment, expression of the polynucleotide coding for a polypeptide whose amino acid sequence is at least 80%, at least 85%, at least 90%, at least 95%, at least 98% or at least 99%, or 100% identical to the amino acid sequence of SEQ ID NO:2 and preferably with the activity of a transhydrogenase is increased by one or more measures selected from the following group:

a) expression of the gene is under the control of a promoter, which in the microorganism used for the process is stronger than the native, or the original promoter of the gene; examples of promoters preferably usable according to the invention in Corynebacterium glutamicum are described for example in FIG. 1 of the review article by Patek et al. (Journal of Biotechnology 104(1-3), 311-323 (2003)). The variants of the dapA promoter described by Vasicova et al. (Journal of Bacteriology 181, 6188-6191 (1999)), for example the promoter A25, can be used in the same way. Furthermore, the gap promoter of Corynebacterium glutamicum (EP 06007373) can be used. Finally, it is possible to use the adequately known promoters T3, T7, SP6, M13, lac, tac and trc described by Amann et al. (Gene 69(2), 301-315 (1988)) and Amann and Brosius (Gene 40(2-3), 183-190 (1985));
b) increasing the copy number of the gene coding for a polypeptide with the activity of a transhydrogenase;

preferably by inserting the gene in plasmids with increased copy number and/or by integrating the gene into the chromosome of the microorganism in at least one copy;

c) expression of the gene takes place using a ribosome binding site, which in the microorganism used for the process is stronger than the original ribosome binding site of the gene;
d) expression of the gene takes place with optimization of codon usage of the microorganism used for the process;
e) expression of the gene takes place with reduction of mRNA secondary structures in the mRNA transcribed by the gene;
f) expression of the gene takes place with elimination of RNA polymerase terminators in the mRNA transcribed by the gene;
g) expression of the gene takes place using mRNA-stabilizing sequences in the mRNA transcribed by the gene.

The stated measures for increasing expression can be suitably combined. Preferably, expression of the polynucleotide coding for a polypeptide with the activity of a transhydrogenase is increased by combining at least two of the measures selected from the group a), b) and c), especially preferably by combining measures a) and b).

As mentioned above, the present invention comprises a microorganism and a process for producing an organic chemical compound by fermentation containing the steps:

a) culturing the microorganism according to the present invention described above in a suitable medium, obtaining a fermentation broth, and
b) enriching the organic chemical compound in the fermentation broth from a).

Preferably a product in liquid or solid form is produced from the fermentation broth.

As mentioned above, the term microorganism comprises bacteria, yeasts and fungi. Among the bacteria, the genus Corynebacterium and the genus Escherichia may be mentioned in particular.

Within the genus Corynebacterium, strains are preferred that are based on the following species:
Corynebacterium efficiens, for example the type strain DSM44549,
Corynebacterium glutamicum, for example the type strain ATCC13032 or the strain R, and
Corynebacterium ammoniagenes, for example the strain ATCC6871,
   wherein the strains of the species Corynebacterium glutamicum are quite especially preferred.

Some strains of the species Corynebacterium glutamicum are also known in the prior art under other designations. These include for example:
strain ATCC13870, which was designated Corynebacterium acetoacidophilum,
strain DSM20137, which was designated Corynebacterium lilium,
strain ATCC17965, which was designated Corynebacterium melassecola,
strain ATCC14067, which was designated Brevibacterium flavum,
strain ATCC13869, which was designated Brevibacterium lactofermentum, and
strain ATCC14020, which was designated Brevibacterium divaricatum.

The term “Micrococcus glutamicus” was also commonly used for Corynebacterium glutamicum.

Some strains of the species Corynebacterium efficiens were also designated Corynebacterium thermoaminogenes in the prior art, for example the strain FERM BP-1539.

The representatives of the Enterobacteriaceae are preferably selected from the genera Escherichia, Erwinia, Providencia and Serratia. The genera Escherichia and Serratia are especially preferred. In the case of the genus Escherichia, in particular the species Escherichia coli may be mentioned, and in the case of the genus Serratia, in particular the species Serratia marcescens may be mentioned.

The microorganisms or strains (starting strains) used for the measures for overexpression of the polypeptide disclosed in the claims preferably already possess the capacity for enriching the desired organic chemical compound or compounds in the cell or for secreting it or them in the surrounding nutrient medium and accumulating it or them there. In the following, the expression “produced” is also used for this. In particular, the strains used for the measures of overexpression possess the capacity to enrich or accumulate ≧(at least) ≧0.10 g/l, 0.25 g/l, 0.5 g/l, ≧1.0 g/l, ≧1.5 g/l, ≧2.0 g/l, ≧4 g/l or ≧10 g/l of the desired compound in ≦(max.) 120 hours, ≦96 hours, ≦48 hours, ≦36 hours, ≦24 hours or ≦12 hours in the cell or in the nutrient medium, respectively. The starting strains are preferably strains that were produced by mutagenesis and selection, by recombinant DNA techniques or by a combination of both methods.

A person skilled in the art will understand that a microorganism suitable for the measures of the invention can also be obtained by first overexpressing a transhydrogenase in a wild strain, for example in the Corynebacterium glutamicum type strain ATCC 13032 or in the strain ATCC 14067, and then causing the microorganism, by further genetic measures described in the prior art, to produce the desired L-amino acid(s). The mere transformation of the wild type with the stated polynucleotide is not a measure according to the invention.

L-Lysine secreting or producing strains of the species Corynebacterium glutamicum that can serve as starting strain for the microorganisms overexpressing transhydrogenase according to the invention are for example:

Corynebacterium glutamicum DSM13994 described in U.S. Pat. No. 6,783,967, and
Corynebacterium glutamicum DM1933 described in Blombach et al. (Appl Environ Microbiol. 2009 Jan; 75(2): 419-27).

An L-lysine secreting or producing strain of the species Corynebacterium efficiens is for example:

Corynebacterium thermoaminogenes AJ12521 (=FERM BP-3304) described in U.S. Pat. No. 5,250,423.

L-Lysine-producing microorganisms typically possess a feedback-resistant or desensitized aspartate kinase. “Feedback-resistant aspartate kinases” are to be understood as aspartate kinases (LysC) that have, compared to the wild form (wild type), a lower sensitivity to inhibition by mixtures of lysine and threonine or mixtures of AEC (aminoethyl cysteine) and threonine or lysine alone or AEC alone. The genes or alleles coding for these aspartate kinases that are desensitized compared to the wild type are also designated as lysC^{FBR} alleles. In the case of the aspartate kinases of the species Corynebacterium glutamicum, the strain ATCC13032 is the suitable wild type. In the prior art, numerous lysC^{FBR} alleles are described that code for aspartate kinase variants, which possess amino acid exchanges compared to the wild-type protein. In the case of bacteria of the genus Corynebacterium, the lysC gene is also designated as the ask gene. In the case of Enterobacteriaceae, the aspartate kinase encoded by the lysC gene is also designated as aspartokinase III.

A detailed list stating which amino acid exchanges in the aspartate kinase protein of Corynebacterium glutamicum lead to desensitization is given in WO2009141330 among others. Aspartate kinase variants that bear the following amino acid exchanges are preferred, selected from the group: at position 380 of the amino acid sequence L-isoleucine instead of L-threonine and optionally at position 381 L-phenylalanine instead of L-serine, at position 311 L-isoleucine instead of L-threonine and at position 279 L-threonine instead of L-alanine.

An L-methionine secreting or producing strain of the species Corynebacterium glutamicum that can serve as starting strain for the microorganisms overexpressing transhydrogenase according to the invention is for example

Corynebacterium glutamicum DSM 17322 described in WO 2007/011939.

Known representatives of L-tryptophan producing or secreting strains of coryneform bacteria that can serve as starting strain for the microorganisms overexpressing transhydrogenase according to the invention are for example:
Corynebacterium glutamicum K76 (=Ferm BP-1847) described in U.S. Pat. No. 5,563,052,
Corynebacterium glutamicum BPS13 (=Ferm BP-1777) described in U.S. Pat. No. 5,605,818, and
Corynebacterium glutamicum Ferm BP-3055 described in U.S. Pat. No. 5,235,940.

Known representatives of L-valine producing or secreting strains of coryneform bacteria that can serve as starting strain for the microorganisms overexpressing transhydrogenase according to the invention are for example:
Brevibacterium lactofermentum FERM BP-1763 described in U.S. Pat. No. 5,188,948,
   Brevibacterium lactofermentum FERM BP-3007 described in U.S. Pat. No. 5,521,074,
   Corynebacterium glutamicum FERM BP-3006 described in U.S. Pat. No. 5,521,074, and
   Corynebacterium glutamicum FERM BP-1764 described in U.S. Pat. No. 5,188,948.

Known representatives of L-isoleucine producing or secreting strains of coryneform bacteria that can serve as starting strain for the microorganisms overexpressing transhydrogenase according to the invention are for example:

Brevibacterium flavum FERM BP-760 described in U.S. Pat. No. 4,656,135,
Brevibacterium flavum FERM BP-2215 described in U.S. Pat. No. 5,294,547, and
Corynebacterium glutamicum FERM BP-758 described in U.S. Pat. No. 4,656,135,

Known representatives of L-homoserine producing or secreting strains of coryneform bacteria that can serve as starting strain for the microorganisms overexpressing transhydrogenase according to the invention are for example:

Micrococcus glutamicus ATCC 14296 described in U.S. Pat. No. 3,189,526 and
Micrococcus glutamicus ATCC 14297 described in U.S. Pat. No. 3,189,526.

Cadaverine producing or secreting microorganisms are for example described in WO 2007/113127.

Known representatives of L-threonine producing or secreting strains of the genus Escherichia, especially of the species

Escherichia coli that can serve as starting strain for the microorganisms overexpressing transhydrogenase according to the invention are for example:

| | |
|---|---|
| Escherichia coli H4581 | (EP 0 301 572) |
| Escherichia coli KY10935 | (Bioscience Biotechnology and |
| | Biochemistry 61(11): 1877-1882 |
| | (1997)) |
| Escherichia coli VNIIgenetika MG442 | (U.S. Pat. No. 4,278,765) |
| Escherichia coli VNIIgenetika M1 | (U.S. Pat. No. 4,321,325) |
| Escherichia coli VNIIgenetika 472T23 | (U.S. Pat. No. 5,631,157) |
| Escherichia coli BKIIM B-3996 | (U.S. Pat. No. 5,175,107) |
| Escherichia coli kat 13 | (WO 98/04715) |
| Escherichia coli KCCM-10132 | (WO 00/09660) |

Known representatives of L-threonine producing or secreting strains of the genus Serratia, especially of the species Serratia marcescens that can serve as starting strain for the microorganisms overexpressing transhydrogenase according to the invention are for example:
Serratia marcescens HNr21 (Applied and Environmental Microbiology 38(6): 1045-1051 (1979))
Serratia marcescens TLr156 (Gene 57(2-3): 151-158 (1987))
Serratia marcescens T-2000 (Applied Biochemistry and Biotechnology 37(3): 255-265 (1992)).

Known representatives of L-tryptophan producing or secreting strains of the genus Escherichia, especially of the species Escherichia coli that can serve as starting strain for the microorganisms overexpressing transhydrogenase according to the invention are for example:

| | |
|---|---|
| Escherichia coli JP4735/pMU3028 | (U.S. Pat. No. 5,756,345) |
| Escherichia coli JP6015/pMU91 | (U.S. Pat. No. 5,756,345) |
| Escherichia coli SV164(pGH5) | (WO94/08031) |
| E. coli AGX17(pGX44) (NRRL B-12263) | (U.S. Pat. No. 4,371,614) |
| E. coli AGX6(pGX50)aroP (NRRL | (U.S. Pat. No. 4,371,614) |
| B-12264) | |
| Escherichia coli AGX17/pGX50, | (WO97/08333) |
| pACKG4-pps | |
| Escherichia coli ATCC 31743 | (CA1182409) |
| E. coli C534/PD2310, pDM136 | (WO87/01130) |
| (ATCC 39795) | |
| Escherichia coli JB102/p5LRPS2 | (U.S. Pat. No. 5,939,295). |

A known representative of L-homoserine producing or secreting strains of the genus Escherichia, especially of the species Escherichia coli that can serve as starting strain for the microorganisms overexpressing transhydrogenase according to the invention is for example:

| | |
|---|---|
| Escherichia coli NZ10rhtA23/pAL4 | (U.S. Pat. No. 6,960,455). |

Known representatives of L-lysine producing or secreting strains of the genus Escherichia, especially of the species Escherichia coli that can serve as starting strain for the microorganisms overexpressing transhydrogenase according to the invention are for example:
Escherichia coli pDA1/TOC21R (=CNCM 1-167) (FR-A-2511032),
Escherichia coli NRRL B-12199 (U.S. Pat. No. 4,346,170)
Escherichia coli NRRL B-12185 (U.S. Pat. No. 4,346,170).

A detailed list stating which amino acid exchanges in the aspartate kinase III protein of Escherichia coli lead to desensitization to inhibition by L-lysine is given inter alia in EP 0 834 559 A1 on page 3 (lines 29 to 41). An aspartate kinase variant is preferred that contains L-aspartic acid instead of glycine at position 323 of the amino acid sequence and/or L-isoleucine instead of L-methionine at position 318.

A known representative of L-valine producing or secreting strains of the genus Escherichia, especially of the species Escherichia coli that can serve as starting strain for the microorganisms overexpressing transhydrogenase according to the invention is for example:
Escherichia coli AJ11502 (NRRL B-12288) (U.S. Pat. No. 4,391,907).

“Polypeptide with the activity of a transhydrogenase” means an enzyme that is able to convert NADH to NADPH and vice versa, i.e., in the context of the present invention, the term “transhydrogenase” means a nicotinamide dinucleotide transhydrogenase.

Transhydrogenases of the most varied organisms are described in public databases, for example the UniProtKB database (Universal Protein Resource Knowledgebase). The UniProtKB database is maintained by the Uniprot Consortium, to which the European Bioinformatics Institute (EBI, Wellcome Trust, Hinxton, Cambridge, United Kingdom), the Swiss Institute of Bioinformatics (SIB, Centre Medical Universitaire, Geneva, Switzerland) and the Protein Information Resource (PIR, Georgetown University, Washington, D.C., US) belong.

The genes for a transhydrogenase can be isolated from the organisms by means of the polymerase chain reaction (PCR) using suitable primers. Instructions are given in, among others, the “PCR” laboratory manual of Newton and Graham (Spektrum Akademischer Verlag, Heidelberg, Germany, 1994) and in WO 2006/100211, pages 14 to 17.

For the measures of the invention, coding genes from Corynebacteria are preferably used for the transhydrogenase. Especially preferably, genes are used that code for polypeptides with activity of a transhydrogenase, whose amino acid sequence is ≧(at least) 80%, ≧90%, ≧92%, ≧94%, ≧96%, ≧97%, ≧98%, ≧99%, identical to the amino acid sequence selected from SEQ ID NO: 2. During the research that led to the present invention, the transhydrogenase-encoding polynucleotide from Corynebacterium glutamicum was identified.

The sequence of the gene is deposited under Seq ID No.1.

From the chemical standpoint, a gene is a polynucleotide. A polynucleotide that encodes a protein/polypeptides is used here synonymously with the term “gene”.

In a preferred embodiment of the process according to the invention, or of the microorganism according to the invention, the latter overexpresses a gene coding for a polypeptide selected from the following i) to vi):

i) a polypeptide consisting of or containing the amino acid sequence shown in SEQ ID NO: 2;
ii) a polypeptide with an amino acid sequence that is at least 80%, at least 85%, at least 90%, at least 95%, at least 98% or at least 99% identical to the amino acid sequence of i), i.e. wherein at least 80%, at least 85%, at least 90%, at least 95%, at least 98% or at least 99% of the amino acid positions are identical to those of SEQ ID NO: 2;
iii) a polypeptide as described under i) or ii), wherein the polypeptide has the activity of a transhydrogenase;
iv) a polypeptide that has an amino acid sequence containing a deletion, substitution, insertion and/or addition of 1 to 90, 1 to 45, 1 to 23, 1 to 12, 1 to 6 amino acid residues with respect to the amino acid sequence shown in SEQ ID NO: 2;
v) a polypeptide as described under iv), wherein the polypeptide has the activity of a transhydrogenase;
vi) a polypeptide as described under v), wherein the polypeptide has the activity of a transhydrogenase and the deletion, substitution, insertion and/or addition of amino acid residues do not essentially affect the activity.

The percentage identity is preferably calculated over the whole length of the amino acid or nucleic acid region. A number of programs, which are based on a large number of algorithms, are available to a person skilled in the art for sequence comparison. In this connection, the algorithms of Needleman and Wunsch or Smith and Waterman give particularly reliable results. The following are available for sequence alignment: the program Pileup (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software package [Genetics Computer Group, 575 Science Drive, Madison, Wis., USA 53711 (1991)]. The percentages given above for the sequence identity are preferably calculated with the GAP program over the whole sequence region.

Optionally, conservative amino acid exchanges are preferred. In the case of aromatic amino acids, the term conservative exchanges is used when phenylalanine, tryptophan and tyrosine are exchanged for one another. In the case of hydrophobic amino acids, the term conservative exchanges is used when leucine, isoleucine and valine are exchanged for one another. In the case of polar amino acids, the term conservative exchanges is used when glutamine and asparagine are exchanged for one another. In the case of basic amino acids, the term conservative exchanges is used when arginine, lysine and histidine are exchanged for one another. In the case of acidic amino acids, the term conservative exchanges is used when aspartic acid and glutamic acid are exchanged for one another. In the case of amino acids containing hydroxyl groups, the term conservative exchanges is used when serine and threonine are exchanged for one another.

Especially preferred configurations of the process according to the invention therefore relate to the production of L-lysine or L-methionine by fermentation using a microorganism of the genus Corynebacterium or Escherichia, characterized in that the following steps are carried out
a) fermentation of a microorganism producing an organic chemical compound, wherein a polynucleotide is overexpressed in the microorganism, wherein the polynucleotide codes for a polypeptide according to SEQ ID NO:2 including 1 to 12, 1 to 6 deletions, substitutions, insertions and/or additions, and wherein the fermentation takes place in a fermentation medium, with formation of a fermentation broth;
b) enrichment of the organic chemical compound in the fermentation broth from a).

Especially preferred configurations of the process according to the invention further relate to the production of L-lysine or L-methionine by fermentation using a microorganism of the genus Corynebacterium or Escherichia, characterized in that the following steps are carried out
a) fermentation of a microorganism producing an organic chemical compound, wherein a polynucleotide is overexpressed in the microorganism, wherein the polynucleotide codes for a polypeptide according to SEQ ID NO:2 including 1 to 12, 1 to 6 deletions, substitutions, insertions and/or additions, wherein the polypeptide has the activity of a transhydrogenase and wherein the fermentation takes place in a fermentation medium, with formation of a fermentation broth;
b) enrichment of the organic chemical compound in the fermentation broth from a).

Furthermore, polynucleotides can be used that hybridize to the nucleotide sequence complementary to SEQ ID NO: 1, preferably to the coding region of SEQ ID NO: 1, under stringent conditions and code for a polypeptide that has the activity of a transhydrogenase.

A person skilled in the art can find instructions for the hybridization of nucleic acids or polynucleotides in, among others, the manual “The DIG System Users Guide for Filter Hybridization” of the company Boehringer Mannheim GmbH (Mannheim, Germany, 1993) and in Liebl et al. (International Journal of Systematic Bacteriology 41: 255-260 (1991)). The hybridization takes place under stringent conditions, i.e. only hybrids are formed for which the probe i.e. a polynucleotide comprising the nucleotide sequence complementary to SEQ ID NO: 1, preferably to the coding region of SEQ ID NO: 1, and the target sequence, i.e. the polynucleotides treated or identified with the probe, are at least 80% identical. It is known that the stringency of the hybridization including the washing steps is influenced or determined by varying the buffer composition, the temperature and the salt concentration. The hybridization reaction is generally carried out at relatively low stringency compared to the washing steps (Hybaid Hybridization Guide, Hybaid Limited, Teddington, UK, 1996).

For example, a buffer corresponding to 5×SSC buffer at a temperature of approx. 50° C.-68° C. can be used for the hybridization reaction. Moreover, probes can also hybridize to polynucleotides that have less than 70% identity to the nucleotide sequence of the probe used. Such hybrids are less stable and are removed by washing under stringent conditions. This can for example be achieved by lowering the salt concentration to 2×SSC or 1×SSC and then optionally 0.5×SSC (The DIG System User's Guide for Filter Hybridization, Boehringer Mannheim, Mannheim, Germany, 1995), setting a temperature of approx. 50° C.-68° C., approx. 52° C.-68° C., approx. 54° C.-68° C., approx. 56° C.-68° C., approx. 58° C.-68° C., approx. 60° C.-68° C., approx. 62° C.-68° C., approx. 64° C.-68° C., approx. 66° C.-68° C. Temperature ranges of approx. 64° C.-68° C. or approx. 66° C.-68° C. are preferred. Optionally, it is possible to lower the salt concentration to a concentration corresponding to 0.2×SSC or 0.1×SSC. Optionally, the SSC buffer contains sodium dodecyl sulphate (SDS) at a concentration of 0.1%. By gradually raising the hybridization temperature in steps of approx. 1-2° C. from 50° C. to 68° C., polynucleotide fragments can be isolated that have at least 80%, at least 90%, at least 92%, at least 94%, at least 96%, at least 97%, at least 98% or at least 99%, optionally 100% identity to the sequence or complementary sequence of the probe used and code for a polypeptide that has the activity of a transhydrogenase. Further instructions for hybridization are commercially available in the form of so-called kits (e.g. DIG Easy Hyb from the company Roche Diagnostics GmbH, Mannheim, Germany, Catalogue No. 1603558).

For the measures of the invention, a gene coding for a transhydrogenase is overexpressed in a microorganism or starting or parent strain producing the desired amino acid(s).

Overexpression is generally understood as an increase in the intracellular concentration or activity of a ribonucleic acid, of a protein (polypeptide) or of an enzyme compared to the starting strain (parent strain) or wild-type strain, when the latter is the starting strain. A starting strain (parent strain) is understood as the strain on which the measure leading to overexpression was carried out.

For overexpression, the methods of recombinant overexpression are preferred. This covers all methods in which a microorganism is produced using a DNA molecule that is prepared in vitro. DNA molecules of this kind comprise for example promoters, expression cassettes, genes, alleles, coding regions etc. These are transferred into the desired microorganism by methods of transformation, conjugation, transduction or similar methods.

Through the measures of overexpression, the activity (total activity in the cell) or concentration of the corresponding polypeptide is generally increased by at least 10%, 25%, 50%, 75%, 100%, 150%, 200%, 300%, 400% or 500%, preferably at most up to 1000%, 2000%, 4000%, 10000% or 20000% relative to the activity or concentration of the polypeptide in the strain before the measure leading to overexpression.

A great many methods are available in the prior art for achieving overexpression. These include increasing the copy number and modifying the nucleotide sequences that regulate or control expression of the gene. The transcription of a gene is controlled inter alia by the promoter and optionally by proteins that repress transcription (repressor proteins) or promote transcription (activator proteins). The translation of the RNA formed is controlled inter alia by the ribosome binding site and the start codon. Polynucleotides or DNA molecules that comprise a promoter and a ribosome binding site and optionally a start codon are also called an expression cassette.

The copy number can be increased by plasmids, which replicate in the cytoplasm of the microorganism. For this, a whole range of plasmids for the most varied groups of microorganisms are described in the prior art, with which the desired increase in the copy number of the gene can be brought about. Suitable plasmids for the genus Escherichia are described for example in the manual Molecular Biology, Labfax (Ed.: T. A. Brown, Bios Scientific, Oxford, UK, 1991). Suitable plasmids for the genus Corynebacterium are described for example in Tauch et al. (Journal of Biotechnology 104 (1-3), 27-40, (2003)), or in Stansen et al. (Applied and Environmental Microbiology 71, 5920-5928 (2005)).

Furthermore, the copy number can be increased by at least one (1) copy by inserting further copies into the chromosome of the microorganism. Suitable methods for the genus Corynebacterium are described for example in the patent documents WO 03/014330, WO 03/040373 and WO 04/069996. Suitable methods for the genus Escherichia are for example the incorporation of a gene copy into the att-site of the phage (Yu and Court, Gene 223, 77-81 (1998)), chromosomal amplification by the Mu phage, as described in EP 0 332 448, or the methods of gene exchange by means of conditionally replicating plasmids described by Hamilton et al. (Journal of Bacteriology 174, 4617-4622 (1989)) or Link et al. (Journal of Bacteriology 179, 6228-6237 (1997)).

The increase in gene expression can moreover be achieved by using a strong promoter, which is linked functionally with the gene to be expressed. Preferably a promoter is used that is stronger than the natural promoter, i.e. that is present in the wild type or parent strain. A whole range of methods are available for this in the prior art.

“Functional linkage” means in this context the sequential arrangement of a promoter with a gene, which leads to expression of the gene and control thereof.

Suitable promoters for the genus Corynebacterium can be found inter alia in Morinaga et al. (Journal of Biotechnology 5, 305-312, (1987)), in the patent documents EP 0 629 699 A2, US 2007/0259408 A1, WO 2006/069711, EP 1 881 076 A1 and EP 1 918 378 A1 and in synoptic accounts such as the “Handbook of Corynebacterium glutamicum” (Eds.: Lothar Eggeling and Michael Bott, CRC Press, Boca Raton, US (2005)) or the book “Corynebacteria, Genomics and Molecular Biology” (Ed.: Andreas Burkovski, Caister Academic Press, Norfolk, UK (2008)). Promoters that can be used are also described in FIG. 1 of the review article by Patek et al. (Journal of Biotechnology 104(1-3), 311-323 (2003)). Similarly, the variants of the dapA promoter described by Vasicova et al. (Journal of Bacteriology 181, 6188-6191 (1999)), for example the promoter A25, can be used. Furthermore, the gap promoter of Corynebacterium glutamicum (EP 06007373, EP 2386650) or variants of the gap promoter (WO 2013000827) can be used. Finally, it is possible to use the adequately known promoters T3, T7, SP6, M13, lac, tac and trc described by Amann et al. (Gene 69(2), 301-315 (1988)) and Amann and Brosius (Gene 40(2-3), 183-190 (1985)). Examples of promoters that permit controlled, i.e. inducible or repressible, expression are described for example in Tsuchiya and Morinaga (Bio/Technology 6, 428-430 (1988)).

Such promoters or expression cassettes are typically inserted at a distance of 1 to 1000, preferably 1 to 500, nucleotides upstream of the first nucleotide of the start codon of the coding region of the gene.

It is also possible to position several promoters before the desired gene or link them functionally with the gene to be expressed and in this way obtain increased expression. Examples of this are described in the patent document WO 2006/069711.

The structure of promoters of Escherichia coli is well known. It is therefore possible to increase the strength of a promoter by modifying its sequence by means of one or more exchange(s) and/or one or more insertion(s) and/or one or more deletion(s) of nucleotides. Examples of this can be found inter alia in “Herder Lexikon der Biologie” [Herder dictionary of biology] (Spektrum Akademischer Verlag, Heidelberg, Germany (1994)).

Examples of the alteration of promoters for increasing expression in coryneform bacteria can be found in U.S. Pat. No. 6,962,805 B2 and in a work by Vasicová et al. (Bacteriol. 1999 October; 181(19): 6188-91.). The strengthening of a target gene by addition or substitution of a homologous promoter can be found for example in EP 1 697 526 B1.

The structure of the ribosome binding site of Corynebacterium glutamicum is also well known and is described for example in Amador (Microbiology 145, 915-924 (1999)) and in genetics handbooks and textbooks, for example “Genes and Clones” (Winnacker, Verlag Chemie, Weinheim, Germany (1990)) or “Molecular Genetics of Bacteria” (Dale and Park, Wiley and Sons Ltd., Chichester, UK (2004)).

To achieve overexpression it is also possible to increase the expression of activator proteins or to reduce or switch off the expression of repressor proteins.

The aforesaid measures for overexpression can be suitably combined. For example, the use of a suitable expression cassette can be combined with increasing the copy number and preferably the use of a suitable promoter can be combined with increasing the copy number.

Instructions for manipulating DNA, digestion and ligation of DNA, transformation and selection of transformants can be found inter alia in the well-known manual of Sambrook et al. “Molecular Cloning: A Laboratory Manual”, Second Edition (Cold Spring Harbor Laboratory Press, 1989).

The extent of expression or overexpression can be found by measuring the amount of mRNA transcribed by the gene, by determining the amount of the polypeptide and by determining the enzyme activity.

For determining the amount of mRNA, it is possible to use, among others, the method of “Northern blotting” and quantitative RT-PCR. In quantitative RT-PCR, the polymerase chain reaction is preceded by reverse transcription. For this, it is possible to use the LightCycler™ system from the company Roche Diagnostics (Boehringer Mannheim GmbH, Roche Molecular Biochemicals, Mannheim, Germany), described for example in Jungwirth et al. (FEMS Microbiology Letters 281, 190-197 (2008)). The concentration of the protein can be determined in the gel by 1- and 2-dimensional protein gel separation followed by optical identification of the protein concentration with corresponding evaluation software. A usual method for preparation of protein gels in the case of coryneform bacteria and for identification of the proteins is the procedure described by Hermann et al. (Electrophoresis, 22: 1712-23 (2001)). The protein concentration can also be determined by Western-blot hybridization with a specific antibody for the protein to be detected (Sambrook et al., Molecular Cloning: A Laboratory Manual. 2^{nd} Ed. Cold Spring Harbor Laboratory Press, Cold Spring Harbor, N.Y., 1989) followed by optical evaluation with appropriate software for determination of concentration (Lohaus and Meyer (1998) Biospektrum 5: 32-39; Lottspeich, Angewandte Chemie 321: 2630-2647 (1999)).

The process according to the invention, and/or the microorganisms according to the invention can be carried out/cultured continuously—as described for example in WO 05/021772—or discontinuously in the batch process (or batch culture) or in the fed-batch or repeated fed-batch process for the purpose of producing the desired organic chemical compound. A summary of a general nature of known culture methods can be found in Chmiel's textbook (Bioprocess Technology 1. Introduction to Bioprocess Technology (Gustav Fischer Verlag, Stuttgart, 1991)) or in the textbook by Storhas (Bioreactors and Peripheral Devices (Vieweg Verlag, Braunschweig/Wiesbaden, 1994)).

The culture medium or fermentation medium to be used must suitably satisfy the requirements of the respective strains. Descriptions of culture media for various microorganisms are given in the “Manual of Methods for General Bacteriology” of the American Society for Bacteriology (Washington D.C., USA, 1981). The terms culture medium and fermentation medium or medium are interchangeable.

A special medium is not required for carrying out the present invention.

Sugars and carbohydrates, for example glucose, sucrose, lactose, fructose, maltose, molasses, sucrose-containing solutions from sugar beet or sugar cane processing, starch, starch hydrolysate and cellulose, oils and fats, for example soya oil, sunflower oil, peanut oil and coconut oil, fatty acids, for example palmitic acid, stearic acid and linoleic acid, alcohols such as for example glycerol, methanol and ethanol and organic acids, for example acetic acid or lactic acid, can be used as the carbon source.

Organic nitrogen-containing compounds such as peptones, yeast extract, meat extract, malt extract, corn steep liquor, soybean flour and urea or inorganic compounds such as ammonium sulphate, ammonium chloride, ammonium phosphate, ammonium carbonate and ammonium nitrate, can be used as the nitrogen source. The nitrogen sources can be used individually or as a mixture.

Phosphoric acid, potassium dihydrogen phosphate or dipotassium hydrogen phosphate or the corresponding sodium-containing salts can be used as the phosphorus source.

The culture medium must moreover contain salts, for example in the form of chlorides or sulphates of metals such as for example sodium, potassium, magnesium, calcium and iron, for example magnesium sulphate or iron sulphate, which are necessary for growth. Finally, essential growth substances such as amino acids, for example homoserine, and vitamins, for example thiamine, biotin or pantothenic acid, can be used in addition to the aforementioned substances.

The aforesaid feed materials can be added to the culture in the form of a single preparation or can be added in a suitable way during culture.

For pH control of the culture, basic compounds such as sodium hydroxide, potassium hydroxide, ammonia or ammonia water or acid compounds such as phosphoric acid or sulphuric acid are used in a suitable manner. The pH is generally adjusted to a value of 6.0 to 8.5, preferably 6.5 to 8. To control the formation of foam, antifoaming agents, for example fatty acid polyglycol esters can be used. To maintain plasmid stability, suitable substances with selective action, for example antibiotics, can be added to the medium. Fermentation is preferably carried out under aerobic conditions. To maintain these conditions, oxygen or oxygen-containing gas mixtures, for example air, are fed into the culture. It is also possible to use liquids that are enriched with hydrogen peroxide. Optionally, fermentation is carried out at excess pressure, for example at an excess pressure of 0.03 to 0.2 MPa. The temperature of the culture is normally 20° C. to 45° C. and preferably 25° C. to 40° C., especially preferably 30° to 37° C. In batch processes, culture is preferably continued until an amount has formed that is sufficient for the measure for obtaining the desired organic chemical compound. This aim is normally achieved within 10 hours to 160 hours. In continuous processes, longer cultivation times are possible. Through the activity of the microorganisms, there is enrichment (accumulation) of the organic chemical compound in the fermentation medium and/or in the cells of the microorganisms.

Examples of suitable fermentation media can be found inter alia in the patent documents 5,770,409, U.S. Pat. No. 5,990,350, U.S. Pat. No. 5,275,940, WO 2007/012078, U.S. Pat. No. 5,827,698, WO 2009/043803, U.S. Pat. No. 5,756,345 or U.S. Pat. No. 7,138,266.

The analysis of L-amino acids for determining the concentration at one or more time point(s) in the course of fermentation can be carried out by separation of the L-amino acids by means of ion exchange chromatography, preferably cation exchange chromatography, with subsequent post-column derivatization using ninhydrin, as described in Spackman et al. (Analytical Chemistry 30: 1190-1206 (1958)). Instead of ninhydrin, ortho-phthadialdehyde can also be used for post-column derivatization. A review article on ion exchange chromatography can be found in Pickering (LC·GC (Magazine of Chromatographic Science) 7(6), 484-487 (1989)).

It is also possible to undertake a pre-column derivatization for example using ortho-phthalaldehyde or phenyl isothiocyanate and separate the resultant amino acid derivatives by reversed-phase (RP) chromatography preferably in the form of high-performance liquid chromatography (HPLC). Such a method is described for example in Lindroth et al. (Analytical Chemistry 51: 1167-1174 (1979)).

Detection is photometric (absorption, fluorescence).

A synoptic account of amino acid analysis can be found inter alia in the textbook “Bioanalytik” by Lottspeich and Zorbas (Spektrum Akademischer Verlag, Heidelberg, Germany 1998).

The analysis of α-keto acids such as KIC for determining the concentration at one or more time point(s) in the course of fermentation can be carried out by separation of the keto acids and other secreted products by means of ion exchange chromatography, preferably cation exchange chromatography on a sulphonated styrene-divinylbenzene polymer in the H+ form, e.g. using 0.025 N sulphuric acid with subsequent UV detection at 215 nm (alternatively also at 230 or 275 nm). Preferably, a REZEX RFQ—Fast Fruit H+ column (Phenomenex) can be used, other suppliers are possible for the separating phase (e.g. Aminex from BioRad). Similar separations are described in corresponding examples of application given by the suppliers.

The performance of the methods or fermentation processes according to the invention with respect to one or more of the parameters selected from the group of concentration (compound formed per volume), yield (compound formed per carbon source consumed), formation (compound formed per volume and time) and specific formation (compound formed per cell dry matter or bio-dry matter and time, or compound formed per cell protein and time) or also other process parameters and combinations thereof, is increased by at least 0.5%, at least 1%, at least 1.5% or at least 2%, relative to methods or fermentation processes with microorganisms in which there is increased activity of a transhydrogenase.

Through the fermentation measures, a fermentation broth is obtained that contains the desired organic chemical compound, preferably L-amino acid.

This is followed by providing or producing or obtaining a product in liquid or solid form containing the organic chemical compound.

A fermentation broth is to be understood as a fermentation medium or nutrient medium in which a microorganism has been cultured for a certain time and at a certain temperature. The fermentation medium or the media used during the fermentation contains/contain all substances or components that ensure production of the desired compound and typically multiplication or viability.

On completion of fermentation, the resultant fermentation broth accordingly contains
a) the biomass (cell mass) of the microorganism resulting from multiplication of the cells of the microorganism,
b) the desired organic chemical compound formed in the course of the fermentation,
c) the organic by-products formed in the course of the fermentation, and
d) the constituents of the fermentation medium used or of the feed materials, for example vitamins such as biotin or salts such as magnesium sulphate, not consumed by the fermentation.

The organic by-products include substances that are produced and optionally are secreted by the microorganisms used in the fermentation, in addition to the respective desired compound. This also includes sugars, for example trehalose.

The fermentation broth is taken from the culture vessel or the fermentation tank, optionally collected, and used for preparing a product containing the organic chemical compound, preferably a product containing L-amino acid, in liquid or solid form. The expression “obtaining the L-amino acid-containing product” is also used for this. In the simplest case, the L-amino acid-containing fermentation broth removed from the fermentation tank is itself the product obtained.

Through one or more of the measures selected from the group
a) partial (>0% to <80%) to complete (100%) or almost complete (≧80%, ≧90%, ≧95%, ≧96%, ≧97%, ≦98%, ≧99%) removal of the water,
b) partial (>0% to <80%) to complete (100%) or almost complete (≧80%, ≧90%, ≧95%, ≧96%, ≧97%, ≧98%, ≧99%) removal of the biomass, wherein this is optionally inactivated before removal,
c) partial (>0% to <80%) to complete (100%) or almost complete (≧80%, ≧90%, ≧95%, ≧96%, ≧97%, ≧98%, ≧99%, ≧99.3%, ≧99.7%) removal of the organic by-products formed in the course of the fermentation, and
d) partial (>0%) to complete (100%) or almost complete (≧80%, ≧90%, ≧95%, ≧96%, ≧97%, ≧98%, ≧99%, ≧99.3%, ≧99.7%) removal of the constituents of the fermentation medium used or of the feed materials not consumed by the fermentation,
   from the fermentation broth, concentration or purification of the desired organic chemical compound is achieved. In this way, products are isolated that have a desired content of the compound.

The partial (>0% to <80%) to complete (100%) or almost complete (≧80% to <100%) removal of water (measure a)) is also termed drying.

In one variant of the process, by complete or almost complete removal of water, of the biomass, of the organic by-products and of the unconsumed constituents of the fermentation medium used, pure (≧80 wt %, ≧90 wt %) or high-purity (≧95 wt %, ≧97 wt %, ≧99% wt %) product forms of the desired organic chemical compound, preferably L-amino acids, are obtained. For the measures according to a), b), c) or d), a whole range of technical instructions are available in the prior art.

In the case of processes for producing organic acids or L-valine, L-leucine and L-isoleucine using bacteria of the genus Corynebacterium, those processes are preferred in which products are obtained that do not contain any constituents of the fermentation broth. These are used in particular in human medicine, in the pharmaceutical industry and in the food industry.

In the case of the amino acid L-lysine, essentially four different product forms are known in the prior art.

One group of L-lysine-containing products comprises concentrated, aqueous, alkaline solutions of purified L-lysine (EP-B-0534865). Another group, as described for example in U.S. Pat. No. 6,340,486 and U.S. Pat. No. 6,465,025, comprises aqueous, acidic, biomass-containing concentrates of L-lysine-containing fermentation broths. The best-known group of solid products comprises pulverulent or crystalline forms of purified or pure L-lysine, which typically is in the form of a salt, for example L-lysine monohydrochloride. Another group of solid product forms is described for example in EP-B-0533039. The product form described there contains, along with L-lysine, most of the feed materials used and not consumed during the production by fermentation and optionally the biomass of the microorganism used with a proportion of >0%-100%.

Corresponding to the various product forms, the most varied processes are known, in which the L-lysine-containing product or the purified L-lysine is prepared from the fermentation broth.

For preparing solid, pure L-lysine, essentially methods of ion exchange chromatography are applied, optionally using activated charcoal and crystallization techniques. In this way, the corresponding base is obtained or a corresponding salt, for example the monohydrochloride (Lys-HCl) or lysine sulphate (Lys₂-H₂SO₄).

A process for producing aqueous, basic L-lysine-containing solutions from fermentation broths is described in EP-B-0534865. In the process described there, the biomass is separated from the fermentation broth and discarded. The pH is adjusted to between 9 and 11 by means of a base, for example sodium, potassium or ammonium hydroxide. After concentration and cooling, the mineral constituents (inorganic salts) are separated from the broth by crystallization and either used as fertilizer or discarded.

In the case of processes for producing lysine using bacteria of the genus Corynebacterium, those processes are preferred in which products are obtained that contain constituents of the fermentation broth. These are used in particular as animal feed additives.

Depending on the requirements, the biomass can be removed completely or partially from the fermentation broth by separation techniques, for example centrifugation, filtration, decanting or a combination thereof, or can be left in it completely. Optionally, the biomass or the fermentation broth containing biomass is inactivated during a suitable process step for example by thermal treatment (heating) or by addition of acid.

In one procedure, the biomass is removed completely or almost completely, so that no (0%) or at most 30%, at most 20%, at most 10%, at most 5%, at most 1% or at most 0.1% of biomass remains in the resultant product. In another procedure the biomass is not removed or is only removed in small proportions, so that all (100%) or more than 70%, 80%, 90%, 95%, 99% or 99.9% of biomass remains in the resultant product. In a process according to the invention, accordingly the biomass is removed in proportions ≦0% to ≦100%.

Finally, the fermentation broth obtained after the fermentation can be adjusted with an inorganic acid, for example hydrochloric acid, sulphuric acid or phosphoric acid or an organic acid, for example propionic acid, to an acidic pH, before or after the complete or partial removal of the biomass (GB 1,439,728 or EP 1 331 220). It is also possible to acidify the fermentation broth with the completely contained biomass. Finally, the broth can also be stabilized by adding sodium bisulphite (NaHSO₃, GB 1,439,728) or some other salt, for example ammonium, alkali-metal or alkaline-earth salt of sulphurous acid.

During separation of the biomass, optionally organic or inorganic solids contained in the fermentation broth are removed partially or completely. The organic by-products dissolved in the fermentation broth and the dissolved, unconsumed constituents of the fermentation medium (feed materials) remain in the product at least partially (>0%), preferably to at least 25%, especially preferably to at least 50% and quite especially preferably to at least 75%. Optionally these also remain completely (100%) or almost completely, i.e. >95% or >98% or above 99%, in the product. If a product contains in this sense at least a proportion of the constituents of the fermentation broth, it is also described with the term “product based on fermentation broth”.

Then the broth is dewatered using known methods, for example using a rotary evaporator, thin-film evaporator, falling-film evaporator, by reverse osmosis or by nanofiltration, or thickened or concentrated. This concentrated fermentation broth can then be processed by methods of freeze-drying, spray-drying, spray granulation or by other methods, for example in the circulating fluidized bed described according to PCT/EP2004/006655, to free-flowing products, especially to a finely-divided powder or preferably coarse-grained granules. Optionally a desired product is isolated from the granules obtained by sieving or dust separation.

It is also possible to dry the fermentation broth directly, i.e. without prior concentration by spray-drying or spray granulation.

“Free-flowing” refers to powders which, from a series of glass discharge vessels with outlet openings of different sizes, flow out unimpeded at least from the vessel with the 5 mm (millimetre) opening (Klein: Seifen, Üole, Fette, Wachse 94, 12 (1968)).

“Finely-divided” means a powder mainly (>50%) with a grain size from 20 to 200 μm diameter.

“Coarse-grained” means a product mainly (>50%) with a grain size from 200 to 2000 μm diameter.

The grain size can be determined by methods of laser diffraction spectrometry. The corresponding methods are described in the textbook on “Teilchengrössenmessung in der Laborpraxis” [Particle size measurement in laboratory practice] by R. H. Muller and R. Schuhmann, Wissenschaftliche Verlagsgesellschaft Stuttgart (1996) or in the textbook “Introduction to Particle Technology” by M. Rhodes, Publ. Wiley & Sons (1998).

The free-flowing, finely-divided powder can moreover be converted by suitable compaction or granulation techniques into a coarse-grained, free-flowing, storable and largely dust-free product.

The term “dust-free” means that the product only contains small proportions (<5%) of grain sizes below 100 μm diameter.

“Storable”, in the sense of this invention, means a product that can be stored in a dry and cool place for at least one (1) year or longer, preferably at least 1.5 years or longer, especially preferably for two (2) years or longer, without any substantial loss (max. 5%) of the respective amino acid.

The invention further relates to a process that is described in outline in WO 2007/042363 A1. For this, using the fermentation broth obtained according to the invention, from which the biomass has optionally been separated completely or partially, a process is carried out that comprises the following steps:

a) the pH is lowered by adding sulphuric acid to 4.0 to 5.2, especially 4.9 to 5.1, and a molar sulphate/L-lysine ratio of 0.85 to 1.2, preferably 0.9 to 1.0, especially preferably >0.9 to <0.95, is established in the broth, optionally by adding another or several sulphate-containing compound(s) and
b) the resultant mixture is concentrated by dewatering and optionally granulated,
wherein optionally before step a), one or both of the following measures is/are carried out:
c) measuring the molar ratio of sulphate/L-lysine for determining the required amount of sulphate-containing compound(s)
d) adding a sulphate-containing compound selected from the group ammonium sulphate, ammonium hydrogen sulphate and sulphuric acid in corresponding proportions.

Optionally, moreover, before step b), a salt of sulphurous acid, preferably alkali-metal hydrogen sulphite, especially preferably sodium hydrogen sulphite is added at a concentration from 0.01 to 0.5 wt %, preferably 0.1 to 0.3 wt %, especially preferably 0.1 to 0.2 wt % relative to the fermentation broth.

As preferred sulphate-containing compounds in the sense of the aforementioned process steps, we may mention in particular ammonium sulphate and/or ammonium hydrogen sulphate or corresponding mixtures of ammonia and sulphuric acid and sulphuric acid itself.

The sulphate/L-lysine molar ratio V is calculated from the formula: V=2x[SO₄²⁻]/[L-lysine]. This formula takes into account that the SO₄²⁻ anion, or sulphuric acid is divalent. A ratio V=1 means that a Lys₂—H₂SO₄ of stoichiometric composition is present, whereas at a ratio of V=0.9 a 10% sulphate deficit is found, and at a ratio of V=1.1 a 10% sulphate excess is found.

During granulation or compaction it is advantageous to use usual organic or inorganic auxiliaries, or carriers, such as starch, gelatin, cellulose derivatives or similar substances, such as usually find application in food processing or animal-feed processing, such as binders, gelling, or thickening agents, or other substances, for example silicic acids, silicates (EP0743016A) and stearates.

Furthermore, it is advantageous to treat the surface of the granules obtained with oils or fats, as described in WO 04/054381. Oils that can be used are mineral oils, vegetable oils or mixtures of vegetable oils. Examples of such oils are soya oil, olive oil, soya oil/lecithin mixtures. Similarly, silicone oils, polyethylene glycols or hydroxyethylcellulose are also suitable. Treating the surfaces with the aforementioned oils gives increased abrasion resistance of the product and a decrease in the proportion of dust. The oil content in the product is 0.02 to 2.0 wt %, preferably 0.02 to 1.0 wt %, and quite especially preferably 0.2 to 1.0 wt % relative to the total amount of the feed additive.

Products are preferred that have a proportion of ≧97 wt % of a grain size from 100 to 1800 μm or a proportion of ≧95 wt % of a grain size from 300 to 1800 μm diameter. The proportion of dust, i.e. particles with a grain size <100 μm, is preferably >0 to 1 wt %, especially preferably max. 0.5 wt %.

Alternatively, however, the product can also be applied on an organic or inorganic carrier that is known and usual in animal-feed processing, for example silicic acids, silicates, grist, bran, flour, starches, sugars or others and/or mixed and stabilized with usual thickeners or binders. Practical examples and processes for this are described in the literature (Die Mühle+Mischfuttertechnik 132 (1995) 49, page 817).

Finally, by coating with film-forming agents, for example metal carbonates, silicic acids, silicates, alginates, stearates, starches, gums and cellulose ethers, as described in DE-C-4100920, the product can also be brought to a state in which it is stable against digestion in animal stomachs, especially the stomach of ruminants.

To establish a desired L-lysine concentration in the product, depending on requirements, the L-lysine can be added during the process in the form of a concentrate or optionally a substantially pure substance or a salt thereof in liquid or solid form. These can be added individually or as mixtures to the fermentation broth obtained or concentrated, or also during the drying or granulation process.

The invention further relates to a process for producing a solid lysine-containing product, which is described in outline in US 20050220933. In this case, using the fermentation broth obtained according to the invention, a process is carried out that comprises the following steps:

a) filtering the fermentation broth, preferably with a membrane filter, so that a biomass-containing sludge and a filtrate are obtained,
b) concentrating the filtrate, preferably so that a solids content from 48 to 52 wt % is obtained,
c) granulating the concentrate obtained in step b), preferably at a temperature from 50° C. to 62° C., and
d) coating the granules obtained in c) with one or more of the coating agent(s)

The concentrating of the filtrate in step b) can also be carried out in such a way that a solids content from 52 to 55 wt %, from 55 to 58 wt % or 58 to 61 wt % is obtained.

For the coating in step d), preferably coating agents are used that are selected from the group consisting of

d1) the biomass obtained in step a),
d2) an L-lysine-containing compound, preferably selected from the group L-lysine hydrochloride or L-lysine sulphate,
d3) an essentially L-lysine-free substance with L-lysine content <1 wt %, preferably <0.5 wt %, preferably selected from the group starch, carrageenan, agar, silicic acids, silicates, grist, bran and flour, and
d4) a water-repelling substance, preferably selected from the group oils, polyethylene glycols and liquid paraffins.

With the measures corresponding to steps d1) to d4), especially d1) to d3), the content of L-lysine is adjusted to a desired value.

In the production of L-lysine-containing products, the ratio of the ions is preferably adjusted so that the molar ratio of the ions according to the following formula

2x[SO₄²⁻]+[Cl⁻]—[NH₄⁺]—[Na⁺]—[K⁺]—2x[Mg²⁺]−2x[Ca²⁺]/[L-Lys]

comes to 0.68 to 0.95, preferably 0.68 to 0.90, especially preferably 0.68 to 0.86, as described by Kushiki et al. in US 20030152633.

In the case of L-lysine, the solid product prepared in this way on the basis of fermentation broth has a lysine content (as lysine base) from 10 wt % to 70 wt % or 20 wt % to 70 wt %, preferably 30 wt % to 70 wt % and quite especially preferably from 40 wt % to 70 wt % relative to the dry matter of the product. Maximum contents of lysine base of 71 wt %, 72 wt %, 73 wt % are also possible.

The water content of the L-lysine-containing solid product is up to 5 wt %, preferably up to 4 wt %, and especially preferably less than 3 wt %.

Strain DM1547 was deposited on 16 Jan. 2001 at the Deutsche Sammlung für Mikroorganismen and Zellkulturen [German Collection for Microorganisms and Cell Cultures] under access number DSM13994.

### DESCRIPTION OF THE FIGURES

FIG. 1 shows plasmid pK18msb_Pg3_lpdA.

FIG. 2 shows plasmid pZ8-1::lpdA.

### EXAMPLES

### Example 1

The gene product of the lpdA gene (cg0790) from Corynebacterium glutamicum was, as in the case of the LpdA protein from Mycobacterium tuberculosis (Argyrou et al., 2004), classified in a predominantly automatic genome annotation as possible dihydrolipoamide dehydrogenase belonging to the flavoprotein disulphide reductase (FDR) family, the members of which have, despite their multiplicity of catalysed redox reactions, a generally high homology with one another at sequence and structure level. The homology of this protein to the already characterized LpdA from Mycobacterium tuberculosis and other flavoprotein disulphide reductases from C. glutamicum and various other organisms was determined by bioinformatic analysis of C. glutamicum LpdA by means of the blastp function of the BLAST program (Basic Local Alignment Search Tool).

### Example 2

### Construction of the Exchange Vector pK18msb_Pg3_lpdA

The nucleotide sequence of the genome of Corynebacterium glutamicum ATCC13032 is described in Ikeda and Nakagawa (Applied Microbiology and Biotechnology 62, 99-109 (2003)), in EP 1 108 790 and in Kalinowski et al. (Journal of Biotechnology 104(1-3), (2003)).

The nucleotide sequence of the genome of Corynebacterium glutamicum is also available in the database of the National Center for Biotechnology Information (NCBI) of the National Library of Medicine (Bethesda, Md., USA), in the DNA Data Bank of Japan (DDBJ, Mishima, Japan) or in the nucleotide sequence database of the European Molecular Biology Laboratories (EMBL, Heidelberg, Germany or Cambridge, UK).

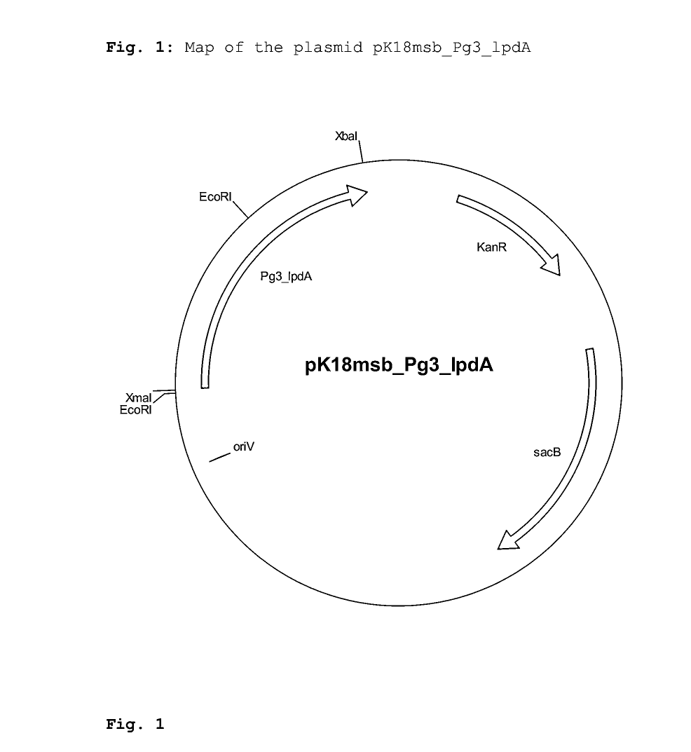

Starting from the genome sequence of Corynebacterium glutamicum ATCC13032, an approx. 1.7-kb DNA fragment was synthesized at the company GeneArt (Regensburg, Germany) (SEQ ID NO: 3), which besides the Pgap3 promoter (DE102011118019.6) bears the flanking sequences for an insertion of the promoter before the native lpdA gene. The fragment was cut via the terminally inserted cleavage sites XmaI and XbaI by XmaI and XbaI cleavage and then cloned into the mobilizable vector pK18mobsacB, also cut with XmaI/XbaI, described by Schäfer et al. (Gene, 145, 69-73 (1994)). The plasmid bears the designation pK18msb_Pg3_lpdA and is shown in FIG. 1.

### Example 3

### Construction of the Vector pZ8-1_lpdA

The lpdA gene from C. glutamicum (cg0790) was amplified using two primers, by which the gene was provided upstream with an EcoRI cleavage site and downstream with a SalI cleavage site. Based on the sequence of the lpdA gene known for C. glutamicum, the following oligonucleotides were used as primers:

lpdA_pZ8-1-1 (SEQ ID NO: 4):
5′ GGTGGTGAATTCAAAGGAGGACAACCATGGCAAAGAGGATCGTA
AT 3′
lpdA_pZ8-1-2 (SEQ ID NO: 5):
5′ GGTGGTGTCGACTTAGCCTAGATCATCATGTT 3′

The primers shown were synthesized by the company MWG Biotech (Ebersberg, Germany).

They make possible the amplification of a 1448-bp-long DNA segment according to SEQ ID NO:6.

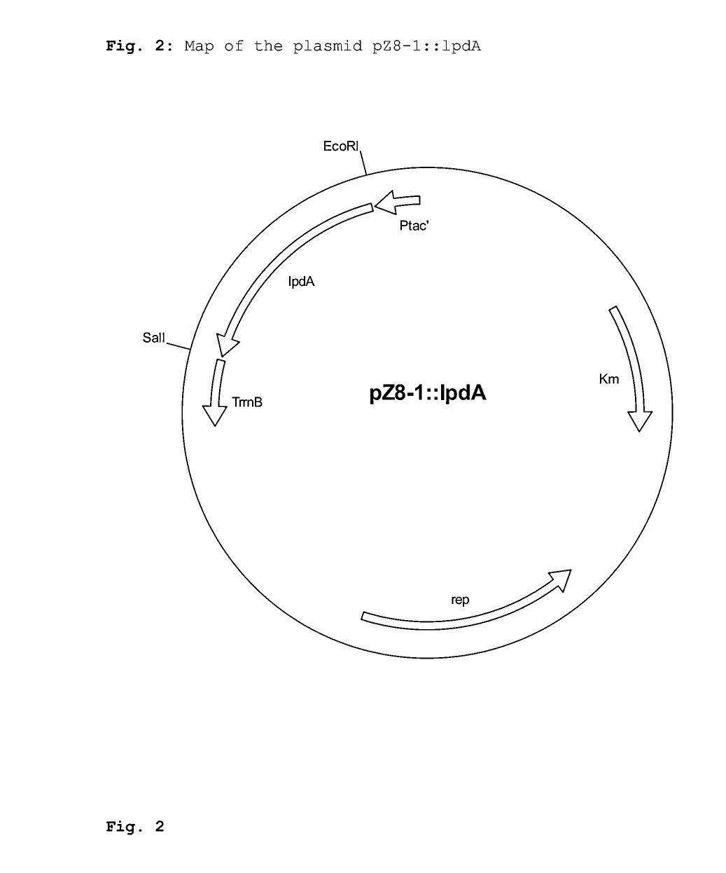

The PCR product was purified with the Nucleospin® Extract II kit. The fragment was cut via the terminally inserted cleavage sites EcoRI and SalI by EcoRI and SalI cleavage and then cloned with the aid of T4 DNA ligase into the vector pZ8-1, also cut by EcoRI/SalI (DE3841454A1; E. coli DH5/pZ8-1 was deposited under number DSM 4939 at the German Collection for Microorganisms). The plasmid bears the designation pZ8-1::lpdA and is shown in FIG. 2.

### Example 4

### Production of the Strain DM1547_Pg3_lpdA

The mutation Pg3_lpdA was to be introduced into the Corynebacterium glutamicum strain DM1547. The strain DM1547 is an aminoethyl cysteine-resistant mutant of Corynebacterium glutamicum ATCC13032. It was deposited under the designation DSM13994 on Jan. 16, 2001 at the German Collection for Microorganisms and Cell Cultures (DSMZ, Braunschweig, Germany).

The vector pK18msb_Pg3_lpdA described in example 2 was electroporated using the electroporation technique of Liebl et al. (FEMS Microbiological Letters, 53: 299-303 (1989)) in Corynebacterium glutamicum DM1547. Selection of plasmid-bearing cells was carried out by plating out the electroporation preparation onto LB agar (Sambrook et al., Molecular Cloning: A Laboratory Manual. 2^{nd} Ed. Cold Spring Harbor Laboratory Press, Cold Spring Harbor, N.Y., 1989), which had been supplemented with 25 mg/l kanamycin. The vector cannot replicate independently in DM1547 and is only retained in the cell when it is integrated in the chromosome as a result of a recombination event. The selection of transconjugants, i.e. of clones with integrated pK18msb_Pg3_lpdA, was carried out by plating out the conjugation preparation on LB agar that had been supplemented with 25 mg/l kanamycin and 50 mg/l nalidixic acid. Kanamycin-resistant transconjugants were then streaked on LB agar plates supplemented with kanamycin (25 mg/l) and incubated at 33° C. for 24 hours. For selecting mutants in which the excision of the plasmid had taken place as a result of a second recombination event, the clones were cultured for 30 hours unselectively in LB liquid medium, then streaked on LB agar that had been supplemented with 10% sucrose and incubated at 33° C. for 24 hours.

The plasmid pK18msb_Pg3_lpdA contains, just like the starting plasmid pK18mobsacB, in addition to the kanamycin-resistance gene, a copy of the sacB gene coding for levansucrase from Bacillus subtilis. The sucrose-inducible expression of the sacB gene leads to the formation of levansucrase, which catalyses the synthesis of the product levan, which is toxic to C. glutamicum.

Therefore only those clones in which the integrated pK18msb_Pg3_lpdA has excised as a result of a second recombination event grow on sucrose-supplemented LB agar. Depending on the position of the second recombination event relative to the mutation site, during excision allele exchange or incorporation of the mutation takes place or the original copy remains in the chromosome of the host.

Then in each case a clone was sought in which the desired exchange, i.e. incorporation of the mutation Pg3_lpdA had taken place.

For this, in each case 20 clones with the phenotype “growth in the presence of sucrose” and “non-growth in the presence of kanamycin” were checked for integration of the mutation Pg3_lpdA using the polymerase chain reaction (PCR).

The following synthetic oligonucleotides (primers) were used for this:

lpdA_1.p (SEQ ID NO: 8):
5′ AACACGTCCCAGGATCAATG 3′
lpdA_2.p (SEQ ID NO: 9):
5′ TTTCGCAAGGTCCTTCACAC 3′

The primers shown were synthesized by the company MWG Biotech (Ebersberg, Germany). The PCR reactions were carried out with the Taq PCR Core Kit from Quiagen (Hilden, Germany), containing the Taq DNA polymerase from Thermus aquaticus, in a Mastercycler from the company Eppendorf (Hamburg, Germany). The conditions in the reaction mixture were set according to the manufacturer's information. The PCR preparation was first submitted to preliminary denaturation at 94° C. for 2 minutes. This was followed by 35×repetition of a denaturation step at 94° C. for 30 seconds, a step for binding the primer to the DNA at 57° C. for 30 seconds and the extension step for lengthening the primer at 72° C. for 60 sec.

After the final extension step for 5 min at 72° C., the PCR preparation was submitted to agarose gel electrophoresis (0.8% agarose). Identification of a DNA fragment with length of 1080 by indicates in this case the integration of the promoter Pg3 before lpdA, whereas a DNA fragment with length of 591 by indicates the wild-type status of the starting strain.

In this way, mutants were identified in which the mutation Pg3_lpdA is present in the form of integration, wherein one of the C. glutamicum strains obtained was designated DM1547_Pg3_lpdA.

### Example 5

### Production of the Strains DM1547/pZ8-1, DM1547/pZ8-1::lpdA, DM1933/pZ8-1 and DM1933/pZ8-1::lpdA

The plasmids pZ8-1 and pZ8-1::lpdA (example 3) were electroporated with the electroporation technique of Liebl et al. (FEMS Microbiological Letters, 53: 299-303 (1989)) into Corynebacterium glutamicum DM1547 and DM1933. Plasmid-bearing cells were selected by plating out the electroporation preparation on LB agar (Sambrook et al., Molecular Cloning: A Laboratory Manual. 2^{nd} Ed. Cold Spring Harbor Laboratory Press, Cold Spring Harbor, N.Y., 1989), which had been supplemented with 25 mg/l kanamycin. Plasmid DNA was isolated from one transformant in each case by the usual methods (Peters-Wendisch et al., 1998, Microbiology, 144, 915-927) and verified by restriction cleavage followed by agarose gel electrophoresis.

The strains obtained were designated DM1547/pZ8-1 and DM1547/pZ8-1::lpdA and DM1933/pZ8-1 and DM1933/pZ8-1::lpdA.

### Example 6

### Production of L-lysine with the Strains DM1547_Pg3_lpdA and DM1547

The C. glutamicum strain DM1547_Pg3_lpdA obtained in example 4 and the starting strain DM1547 were cultured in a nutrient medium suitable for production of lysine and the lysine content in the culture supernatant was determined.

For this, the clones were first multiplied on brain-heart agar plates (Merck, Darmstadt, Germany) for 24 hours at 33° C. Starting from these agar plate cultures, one preculture was inoculated in each case (10 ml medium in a 100-ml Erlenmeyer flask). Medium MM was used as the preculture medium. The preculture was incubated for 24 hours at 33° C. and 240 rpm on a shaker. From this preculture, a main culture was inoculated, so that the initial OD (660 nm) of the main culture was 0.1 OD. Medium MM was also used for the main culture.

Table 1 gives a synopsis of the composition of the culture medium used.

TABLE 1
Medium MM

| | | |
|---|---|---|
| CSL (corn steep liquor) | 5 | g/l |
| MOPS (morpholinopropanesulphonic acid) | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| CaCO₃ | 25 | g/l |

CSL (corn steep liquor), MOPS (morpholinopropanesulphonic acid) and the salt solution were adjusted to pH 7 with ammonia water and autoclaved. Then the sterile substrate and vitamin solutions and the dry autoclaved CaCO₃ were added.

Culture was carried out in volumes of 10 ml, which were contained in 100-ml Erlenmeyer flasks with baffles. The temperature was 33° C., the rotary speed was 250 rpm and the air humidity 80%.

After 40 hours the optical density (OD) was determined at a measurement wavelength of 660 nm with the Biomek 1000 (Beckmann Instruments GmbH, Munich). The amount of lysine formed was determined with an amino acid analyser from the company Eppendorf-BioTronik (Hamburg, Germany) by ion exchange chromatography and post-column derivatization with ninhydrin detection.

The test result is shown in Table 2.

TABLE 2
Production of L-lysine

| Strain | L-lysine HCl (g/l) | OD (660 nm) |
|---|---|---|
| DM1547 | 19.0 | 12.3 |
| DM1547_Pg3_lpdA | 20.8 | 12.2 |

All values are mean values from 3 independent experiments with the stated strains.

The result shows that the yield of the desired product (L-lysine) has clearly increased.

### Example 7

### Production of L-lysine with the Strains DM1547/pZ8-1::lpdA, DM1547/pZ8-1, DM1933/pZ8-1 and DM1933/pZ8-1::lpdA

The C. glutamicum strains DM1547/pZ8-1::lpdA and DM1933/pZ8-1::lpdA obtained in example 5 and the control strains DM1547/pZ8-1 and DM1933/pZ8-1 were cultured in a nutrient medium suitable for production of lysine and the lysine content in the culture supernatant was determined.

For this, first the strains were incubated on an agar plate with the corresponding antibiotic (brain-heart agar with kanamycin (25 mg/l)) at 33° C. for 24 hours. Starting from these agar plate cultures, one preculture was inoculated in each case (10 ml medium in a 100-ml Erlenmeyer flask). Medium MM (Table 1 from example 6) was used as the preculture medium, to which kanamycin (25 mg/l) was added.

The preculture was incubated for 24 hours at 33° C. and 240 rpm on a shaker. From this preculture, a main culture was inoculated, so that the initial OD (660 nm) of the main culture was 0.1 OD. Medium MM was also used for the main culture.

Culture was carried out in volumes of 10 ml, which were contained in 100-ml Erlenmeyer flasks with baffles. The temperature was 33° C., the rotary speed was 250 rpm and the air humidity 80%.

After 40 hours, the optical density (OD) was determined at a measurement wavelength of 660 nm with the Biomek 1000 (Beckmann Instruments GmbH, Munich). The amount of lysine formed was determined with an amino acid analyser from the company Eppendorf-BioTronik (Hamburg, Germany) by ion exchange chromatography and post-column derivatization with ninhydrin detection.

The test result is shown in Table 3.

TABLE 3
Production of L-lysine

| Strain | L-lysine HCl (g/l) | OD (660 nm) |
|---|---|---|
| DM1933/pZ8-1 | 12.2 | 13.1 |
| DM1933/pZ8-1::lpdA | 14.5 | 13.0 |
| DM1547/pZ8-1 | 18.5 | 12.5 |
| DM1547/pZ8-1::lpdA | 21.0 | 12.3 |

All values are mean values from 3 independent experiments with the stated strains.

The result shows that the yield of the desired product (L-lysine) has clearly increased.

### Example 8

### Transformation of the strain ECM3 with the plasmids pZ8-1 and pZ8-1 1pdA

The L-methionine-producing E. coli strain ECM3 is based on the wild-type K12 strain MG1655. The strain ECM3 carries, as described in EP2205754A2, EP2326724A1 and EP12156052.8, a feedback-resistant metA allele, a deletion of the genes metJ, yncA, pykA, pykF and purU, a variant of the spoT gene, and in each case promoter strengthening before the genes metH, metF, gcvT, cysP and cysJ.

The strain ECM3 was transformed with the plasmids pZ8-1 and pZ8-1 1pdA from example 3 and the transformants were selected with 20 mg/l kanamycin. The resulting strains were designated ECM3/pZ8-1 and ECM3/pZ8-1_lpdA.

### Example 9

### Production of L-methionine with the Strains ECM3 and ECM3/pZ8-1_lpdA

The performance of the E. coli L-methionine production strains ECM3 and ECM3/pZ8-1_lpdA was assessed by production tests in 100-ml Erlenmeyer flasks. As precultures, in each case 10 ml of preculture medium (10% LB medium with 2.5 g/l glucose and 90% minimal medium PC1 (Table 4)) was inoculated with 100 μl cell culture and cultured for 10 hours at 37° C. Then in each case 10 ml of PC1 minimal medium was inoculated with this to an OD 600 nm of 0.2 (Eppendorf Bio-Photometer; Eppendorf AG, Hamburg, Germany) and cultured for 24 hours at 37° C. The extracellular L-methionine concentration was determined with an amino acid analyser (Sykam GmbH, Eresing, Germany) by ion exchange chromatography and post-column derivatization with ninhydrin detection. The extracellular glucose concentration was determined with a YSI 2700 Select Glucose Analyzer (YSI Life Sciences, Yellow Springs, Ohio, USA). The results are shown in Table 5. After 48 hours, the glucose in both cultures was completely consumed.

TABLE 4
Minimal Medium PC1

| Substance | Concentration | |
|---|---|---|
| ZnSO₄ * 7 H₂O | 4 | mg/l |
| CuCl₂ * 2 H₂O | 2 | mg/l |
| MnSO₄ * H₂O | 20 | mg/l |
| H₃BO₃ | 1 | mg/l |
| Na₂MoO₄ * 2 H₂O | 0.4 | mg/l |
| MgSO₄ * 7 H₂O | 1 | g/l |
| Citric acid * 1 H₂O | 6.56 | g/l |
| CaCl₂ * 2 H₂O | 40 | mg/l |
| K₂HPO₄ | 8.02 | g/l |
| Na₂HPO₄ | 2 | g/l |
| (NH₄)₂HPO₄ | 8 | g/l |
| NH₄Cl | 0.13 | g/l |
| (NH₄)₂SO₃ | 5.6 | g/l |
| MOPS | 5 | g/l |
| NaOH 10M | adjusted to pH 6.8 | |
| FeSO₄ * 7 H₂O | 40 | mg/l |
| Thiamine hydrochloride | 10 | mg/l |
| Vitamin B12 | 10 | mg/l |
| Glucose | 10 | g/l |
| Kanamycin | 50 | mg/l |

TABLE 5
L-methionine concentrations in the fermentation
broths of the E. coli strains investigated

| | | L-methionine |
|---|---|---|
| Strain | OD(600 nm) | (g/l) |
| ECM3/pZ8-1 | 3.38 | 0.40 |
| ECM3/pZ8-1_lpdA | 3.17 | 0.57 |

All values are mean values from 3 independent experiments with the stated strains.

The result shows that the yield of the desired product (L-methionine) has clearly increased.

FIG. 1: Map of the Plasmid pK18msb_Pg3_lpdA

The abbreviations and designations used have the following meanings. The base pair numbers stated are approximate values, which are obtained within the range of measurement reproducibility.

| | |
|---|---|
| KanR: | Kanamycin resistance gene |
| XmaI | Cleavage site of the restriction enzyme XmaI |
| EcoRI | Cleavage site of the restriction enzyme EcoRI |
| XbaI | Cleavage site of the restriction enzyme XbaI |
| sacB: | sacB gene |
| oriV: | Replication origin V |

FIG. 2: Map of the Plasmid pZ8-1::lpdA

The abbreviations and designations used have the following meanings. The base pair numbers stated are approximate values, which are obtained within the range of measurement reproducibility.

| | |
|---|---|
| Km | Kanamycin resistance gene |
| EcoRI | Cleavage site of the restriction enzyme EcoRI |
| SalII | Cleavage site of the restriction enzyme SalI |
| Ptac | tac-promoter |
| rep | Escherichia coli replication origin |
| TrrnB | rrnB terminator |

1. Process for producing an organic chemical compound by fermentation using a microorganism, characterized in that the following steps are carried out:
a) fermentation of a microorganism producing an organic chemical compound, wherein a polynucleotide that codes for a polypeptide whose amino acid sequence is at least 80% identical to the amino acid sequence of SEQ ID NO:2 is overexpressed in the microorganism and wherein the fermentation takes place in a fermentation medium, with formation of a fermentation broth,
b) enrichment of the organic chemical compound in the fermentation broth from a).

2. Process for producing an organic chemical compound according to claim 1, characterized in that a polynucleotide that codes for a polypeptide whose amino acid sequence is at least 95% identical to the amino acid sequence of SEQ ID NO:2 is overexpressed in the microorganism.

3. Process for producing an organic chemical compound according to claim 1, characterized in that the encoded polypeptide has the activity of a transhydrogenase.

4. Process for producing an organic chemical compound according to claim 1, characterized in that the encoded polypeptide comprises the amino acid sequence of SEQ ID NO:2.

5. Process for producing an organic chemical compound according to claim 1, characterized in that the production of the organic chemical compound is increased by at least 0.5% relative to the fermentation of a microorganism in which the polynucleotide that codes for a polypeptide whose amino acid sequence is at least 80% identical to the amino acid sequence of SEQ ID NO:2 is not overexpressed.

6. Process for producing an organic chemical compound according to claim 1, characterized in that the overexpression is achieved by one or more of the measures selected from the group
a) expression of the gene is under the control of a promoter, which is stronger in the microorganism used for the process than the native promoter of the gene;
b) increase of the copy number of the gene coding for a polypeptide with the activity of a transhydrogenase;
preferably by inserting the gene in plasmids with increased copy number and/or by integrating the gene into the chromosome of the microorganism in at least one copy;
c) expression of the gene takes place using a ribosome binding site, which is stronger in the microorganism used for the process than the original ribosome binding site of the gene;
d) expression of the gene takes place with optimization of the codon usage of the microorganism used for the process;
e) expression of the gene takes place with reduction of mRNA secondary structures in the mRNA transcribed by the gene;
f) expression of the gene takes place with elimination of RNA polymerase terminators in the mRNA transcribed by the gene;
g) expression of the gene takes place using mRNA-stabilizing sequences in the mRNA transcribed by the gene.

7. Process for producing an organic chemical compound according to claim 1, characterized in that the organic chemical compound is an L-amino acid selected from the group L-threonine, L-isoleucine, L-lysine, L-methionine, L-ornithine, L-proline, L-valine, L-leucine and L-tryptophan.

8. Process for producing an organic chemical compound according to claim 1, characterized in that it is an organic chemical compound-secreting microorganism of the genus Corynebacterium or Escherichia.

9. Process for producing an organic chemical compound according to claim 1, characterized in that it is an organic chemical compound-secreting microorganism of the species Corynebacterium glutamicum or of the species Escherichia coli.

10. Process for producing an organic chemical compound according to claim 1, characterized in that it is a process selected from the group: batch process, fed-batch process, repeated fed-batch process and continuous process.

11. Process according to claim 7, characterized in that the organic chemical compound or a liquid or solid organic chemical compound-containing product is obtained from the organic chemical compound-containing fermentation broth.

12. Microorganism that produces an organic chemical compound, in which a polynucleotide that codes for a polypeptide whose amino acid sequence is ≧80% identical to the amino acid sequence of SEQ ID NO: 2 is overexpressed.

13. Microorganism according to claim 12, characterized in that the amino acid sequence of the encoded polypeptide is ≧95% identical to the amino acid sequence of SEQ ID NO: 2.

14. Microorganism according to claim 12, characterized in that the encoded polypeptide comprises the amino acid sequence of SEQ ID NO: 2.

15. Microorganism according to claim 12, selected from the group of the genus Corynebacterium and the bacteria of the family Enterobacteriaceae, wherein the species Corynebacterium glutamicum is preferred.

16. Microorganism according to claim 12, characterized in that the overexpressed polynucleotide has the activity of a transhydrogenase.
